(19) United States
(12) Patent Application Publication
(10) Pub. No.: US 2026/0049290 A1
(43) Pub. Date: Feb. 19, 2026

(54) HUMAN SPINAL CORD INJURY ORGANOIDS

(71) Applicant: Northwestern University, Evanston, IL (US)

(72) Inventors: Nozomu Takata, Evanston, IL (US); Nicholas Allen Sather, Evanston, IL (US); Samuel Isaac Stupp, Evanston, IL (US)

(21) Appl. No.: 19/259,714

(22) Filed: Jul. 3, 2025

Related U.S. Application Data
(63) Continuation-in-part of application No. 18/735,858, filed on Jun. 6, 2024.
(60) Provisional application No. 63/506,521, filed on Jun. 6, 2023.

(51) Int. Cl. C12N 5/071

(52) U.S. Cl. CPC C12N 5/0697; C12N 2503/04; C12N 2506/45

(57) ABSTRACT

Provided herein are engineered human spinal cord organoids and methods of use thereof for development and testing of therapeutic treatment for spinal cord injury.

### PRIORITY STATEMENT

This application is a continuation in part of U.S. application Ser. No. 18/735,858, filed Jun. 6, 2024, which claims priority to U.S. Provisional Application No. 63/506,521, filed Jun. 6, 2023, the entire contents of each of which are incorporated herein by reference for all purposes.

### SEQUENCE LISTING

The text of the computer readable sequence listing filed herewith, titled “NWEST_42124_503_SequenceListing”, created Jul. 2, 2025, having a file size of 8,039 bytes, is hereby incorporated by reference in its entirety.

### FIELD

Provided herein are engineered human spinal cord organoids and methods of use thereof for development and testing of therapeutic treatments for spinal cord injury.

### BACKGROUND

Spinal cord injury (SCI) is the leading cause of permanent disability and death in children and adults with between 250,000 and 500,000 cases per year, resulting in paralysis and causing other serious health complications. Despite extensive research and interest in treating this devastating injury, effective therapies do not currently exist for either acute or chronic SCI. Injury in organs triggers cellular and molecular changes that contribute to endogenous repair processes, whereas the central nervous system (CNS) in humans has poor regeneration capacity. The spinal cord has a more pronounced inflammatory response to traumatic injury than other CNS tissues, which contributes to both tissue damage and repair. The current paradigm for understanding the pathophysiology and potential treatment of SCI relies largely on animal model studies. Therapeutic treatments for SCI thus remain challenging due to two major reasons: limited availability of injured human spinal cord tissues, and lack of experimental models that allow us to learn more rapidly about the injury environment associated with neuroinflammation, and glial scar in humans. Consequently, reliable methods for assessing potential therapies for human SCI are currently not available. These limitations provide great challenges for effectively developing therapeutic drugs to prevent injury progression or to promote regeneration in human spinal cords.

### SUMMARY

In some aspects, provided herein are human spinal cord organoids. In some embodiments, provided herein is a human spinal cord organoid (hSCO) comprising neural cells, astroglial cells, and immune cells. In some embodiments, the immune cells comprise microglia, macrophages, neutrophils, T-cells, or a combination thereof. In some embodiments, the immune cells comprise microglia. In some embodiments, the neural cells express class III beta-tubulin (TUJ-1), the microglia express IBA-1, and the astroglial cells express GFAP. In some embodiments, the hSCO further comprises oligodendrocytes (e.g. myelin-producing cells) and/or ependymal cells.

In some embodiments, the hSCO is substantially spherical in shape and has an average diameter of at least 2 mm. In some embodiments, the hSCO has an average diameter of at least 2.5 mm. In some embodiments, the hSCO has an average diameter of about 3 mm.

In some embodiments, the astroglial cells are present on an outer surface of the organoid, and the outer surface is substantially smooth.

In some embodiments, the hSCO has been cultured for at least about 8 weeks. In some embodiments, the hSCO has been cultured for about 8 weeks to about 28 weeks.

In some embodiments, the hSCO has received an injury and comprises glial scar-like tissue and chondroitin sulfate proteoglycans (CSPGs) as a result of the injury.

In some embodiments, provided herein are methods of simulating spinal cord injury using the hSCOs herein. In some embodiments, provided herein is a method of simulating a spinal cord injury, comprising mechanically injuring a human spinal cord organoid (hSCO). In some embodiments, the hSCO comprises neural cells, astroglial cells, and immune cells. In some embodiments, the hSCO comprises neural cells and astroglial cells, and immune cells or immune cell progenitors are added to and infiltrate the hSCO after mechanical injury.

In some embodiments, the immune cells comprise microglia, macrophages, neutrophils, T-cells, or a combination thereof. In some embodiments, the immune cells comprise microglia. In some embodiments, the neural cells express class III beta-tubulin (TUJ-1), the microglia express IBA-1, and the astroglial cells express GFAP. In some embodiments, the hSCO further comprises oligodendrocytes (e.g. myelin-producing cells) and/or ependymal cells.

In some embodiments, the mechanically injuring the hSCO comprises cutting (e.g. lacerating) the hSCO or applying a compressive impact force to the hSCO. In some embodiments, mechanically injuring the hSCO produces a glial scar-like tissue and chondroitin sulfate proteoglycans (CSPGs) on the hSCO.

In some embodiments, the method further comprises contacting the injured hSCO with a potential therapeutic agent for the treatment of spinal cord injury, and assessing a response of the injured hSCO to the potential therapeutic agent.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1V show identification of neuronal and glial cells in human spinal cord organoids (hSCOs). (FIG. 1A) Schematic illustrating human spinal cord organoid production indicating the appearance of different cell types as a function of time in culture. (FIG. 1B) Light microscopy images of growing organoids at 3, 7, and 14 weeks in culture. (FIG. 1C) Bar graph comparing the average diameter of human organoids versus the mouse spinal cord (error bars correspond to three independent repeat experiments. *P<0.05; **P<0.01; ***P<0.001; one-way ANOVA followed by Tukey's post-hoc test). (FIG. 1D-K) Representative fluorescence micrographs obtained after immunostaining of native mouse spinal cord tissues (FIG. 1D, FIG. IF, FIG. 1H, FIG. 1I) and hSCOs (FIG. 1E, FIG. 1G, FIG. 1J, FIG. 1K) at corresponding developmental times (I and K are high magnifications of insets in H and J, respectively); NESTIN shows neural stem cells in yellow, TUJ-1 neurons in green, GFAP glial cells in magenta and 4,6-diamidino-2-phenylindole (DAPI) nuclei in blue. (FIG. 1L) Schematic illustration of single-cell isolation for single-cell RNA-seq and bioinformatics analysis. (FIG. 1M) t-distributed stochastic neighbor embedding (1-SNE) plot analysis for organoids cultured 5 and 14 weeks. (FIG. 1N) 1-SNE plots of transcriptionally distinguishable clusters by unique colors based on the bioinformatic analysis that partitions the cells sub-types into 12 groups (clusters 0-11). The distinct cell sub-types are defined by the known spinal cord signature gene expression found in human fetuses from available omics data in public. NPC, neural progenitor cell. OPC, oligodendrocyte progenitor cell. Uniform manifold approximation and projection (UMAP) shows the transcriptional difference between each cell subtype found in human organoids. (FIG. 1O-FIG. 1V) Representative micrographs by SEM analysis showing organoid outer-surface and cross-sectional images at each corresponding time (7 weeks, FIG. 1O, FIG. 1P, FIG. 1Q, FIG. 1R. 24 weeks, FIG. 1S, FIG. 1T, FIG. 1U, FIG. 1V) in the organoid culture. P or R is high magnifications of insets in O or Q, respectively. T or V is high magnifications of insets in S or U, respectively. Scale bars, 100 μm (D-K), 200 μm (O, Q, S, U), 20 μm (P, R, T, V).

FIGS. 2A-2P show two injury organoid models. (FIG. 2A, FIG. 2E) Schematic illustration showing the sharp scalpel-mediated injury or impactor-mediated contusion injury. (FIG. 2B, FIG. 2F) Bright-field images of 24-w human spinal cord organoids immediately after SCI. (FIG. 2D, FIG. 2H) Fluorescence microscopic image of acute cell death post-SCI by live and dead staining assay. (FIG. 2C, FIG. 2G) Bar graph of LDH activity, indicating cytotoxicity before and after the injury by specific absorbance. Error bars correspond to three independent repeat experiments. **P<0.01; Unpaired Student's t test (two-tailed) was performed. (FIG. 2I, FIG. 2M) Schematic illustration of a cross-section view of the injured organoid. (FIG. 2J, FIG. 2N) Representative micrographs by immunostaining analysis using antibodies for glia and neuronal markers. (FIG. 2K, FIG. 2L, FIG. 2O, FIG. 2P) High magnification images of insets in I. Scale bars, 1 mm (B-E), 100 μm (J-K), 20 μm (N).

FIGS. 3A-3O show spinal cord neurites were promoted by PA nanostructures with supramolecular motion. (FIG. 3A) Schematic illustration of PA nanofiber treatment in solution for uninjured organoid culture. (FIG. 3B. FIG. 3C, FIG. 3D) Representative fluorescent micrographs showing localization of co-assemble PAs visualized by Alexa 647 dye. (FIG. 3E) Bar graph indicating relative brightness of fluorescently labeled PA localization in uninjured organoids before and after medium wash. Error bars correspond to three independent repeat experiments. **P<0.01; n.s. not significant; One-way ANOVA followed by Tukey's post-hoc test was performed. % indicates the reduction of the fluorescent signals after wash. (FIG. 3F-FIG. 3M) Representative fluorescent images visualized by Fluo-4 dye showing neurite growth in each treated group. FIG. 3G, FIG. 3I, FIG. 3K or FIG. 3M is high magnification of FIG. 3F, FIG. 3H, FIG. 3J or FIG. 3L, respectively. (FIG. 3N) Line graph of neurite length quantification by elapse-time. Error bars correspond to three independent repeat experiments. *P<0.05; **P<0.01; ***P<0.001; One-way ANOVA followed by Tukey's post-hoc test was performed. (FIG. 3O) A polar histogram indicating neurite-growth angle relative to axial direction (perpendicular to the organoid) in PAI and PA2 conditions. Scale bars, 100 nm (B-D), 500 nm (F-M).

FIGS. 4A-4Z show Two injury models treated by PA nanofibers. (FIG. 4A) Schematic illustration of the human spinal cord organoid injury induced by a scalpel and PA treatment. (FIG. 4B, FIG. 4C) Fluorescence micrographs of organoid sections stained for glia (GFAP, magenta) and nuclei (DAPI, blue). (FIG. 4D) Bar graphs indicate quantification of relative GFAP intensity in injured versus uninjured areas. (FIG. 4E, FIG. 4F) Fluorescence micrographs of neurite growth into PA gels. Fluo-4 indicates neurite structure (green) and Alexa Fluor 647 indicates PA gels (blue). (FIG. 4G) Bar graphs indicate neurite length one month after PA treatment. (FIG. 4H-FIG. 4M) SEM analysis on the injured surface of organoids treated with E2PA or IKVAV PA2. (FIG. 4N) Schematic illustration of the impactor-mediated contusion injury and PA treatment. (FIG. 4O, FIG. 4P) Fluorescence micrographs of organoids stained for glia (GFAP, magenta). (FIG. 4Q) Bar graphs indicate quantification of relative GFAP intensity in injured versus uninjured areas. (FIG. 4R, FIG. 4S) Fluorescence micrographs of neurite growth into PA gels. Fluo-4 indicates neurite structure (green) and Alexa Fluor 647 indicates PA gels (blue). (FIG. 4T) Bar graph indicates neurite length one month after PA treatment. (FIG. 4U-FIG. 4Z) SEM analysis on the injured surface of organoids treated with E2 PA or IKVAV PA2. Scale bars, 100 μm (B, C, E, F, K, O, P, R, S, X), 200 μm (H, U). 20 μm (I, L), 10 μm (V, Y), 5 μm (J, M), 2 μm (W, Z). Error bars correspond to standard error of the mean from three independent repeat experiments. Unpaired Student's t test (two-tailed) was performed (*P <0.05, **P<0.01) (D, G, Q, T).

FIGS. 5A-5C show histology of human spinal cord organoids (hSCOs). Representative fluorescence micrographs of hSCOs for 7 weeks or 28 weeks; Immunostaining by specific biomarkers (FIG. 5A) OLIG2 (orange), F-Actin (blue). (FIG. 5B, FIG. 5C) GFAP (magenta), TUJ-1 (green), and DAPI (blue). Scale bars: 500 μm.

FIGS. 6A-6G show characterization of two injured organoid models. (FIG. 6A, FIG. 6B, FIG. 6C, FIG. 6D) Ultrastructural micrographic analysis of organoids injured using a scalpel model after one month culture. (FIG. 6E, FIG. 6F, FIG. 6G) Ultrasonic micrographic analysis of organoids injured using an impactor after one month culture. Scale bars: 20 μm (C, D), and 10 μm (F, G).

FIGS. 7A-7C show live/dead staining of an uninjured organoid. (FIG. 7A) schematic illustration of an uninjured organoid. (FIG. 7B) Bright field image of the organoid (FIG. 7C) Live dead analysis via live (green)-dead (red) assay. Scale bars: 1 mm.

FIG. 8 shows visualization of LAMININ in Matrigel treated sample. Immunostaining using specific LAMININ antibody shows the protein (cyan) localization on organoid surface. Scale bars: 100 μm.

FIGS. 9A-9B show structures of peptide amphiphiles used to treat the injured hSCOs herein.

FIGS. 10A-10S show neuroinflammatory response in injured microglia containing organoids. (FIG. 10A) Schematic illustration of microglia assembly pathway 1 to form the neuroimmune organoid. (FIG. 10B) Representative bright-field micrographs of myeloid progenitors. (FIG. 10C) Schematic illustration of assembly pathway 1 using erythro-myeloid progenitors (EMPs) (Sup1, 2, 3 are supplemental reagents in a microglia maturation kit from Stem Cell Technologies). (FIG. 10D) Fluorescence micrograph of mouse spinal cord (IBA-1, red and DAPI, blue). (FIG. 10E, FIG. 10F) Fluorescence micrographs of human spinal cord (SC) organoids (CellTracker, magenta and organoid background, blue). (FIG. 10G) Fluorescence micrographs of microglia assembly 1 organoid (24 weeks) stained for astroglia (GFAP, green) and microglia (IBA-1, red). (FIG. 10H) Schematic illustration of organoid cells after dissociation for flow cytometry analysis (top); the graph reveals microglia % population of cells positive for both CD11b and CD45 in the human SC organoids (bottom). (FIG. 10I) Bar graph showing the population of cells that are positive for both CD11b and CD45 in organoids with or without EMPs. (FIG. 10J, FIG. 10K) Quantitative PCR analysis of CIQA and C3 associated with inflammation relative to β-ACTIN and 18S, respectively. (FIG. 10L) Representative fluorescence micrographs showing neurite outgrowth from the surface of injured organoids treated with non-bioactive E2 PA or IKVAV PA2 (visualized by Fluo-4 dye added to the medium). (FIG. 10M) Bar graph of neurite length of organoids treated with E2 PA or IKVAV PA2 in panel L. (FIG. 10N) Fluorescence micrographs of axon marker neurofilament protein (yellow) in injured organoid surfaces treated with E2 or IKAVA PAS (DAPI, blue). (FIG. 10O) Schematic illustration showing the regions outlined by the black and red boxes in panels P and R. (FIG. 10P) Fluorescence micrographs of GFAP (magenta) treated with E2 or IKAVA PAS (DAPI, blue). (FIG. 10Q) Bar graphs of relative GFAP intensity in injured versus uninjured areas. (FIG. 10R) Fluorescence micrographs of CSPG (red) treated with E2 or IKAVA PAS (DAPI, blue). (FIG. 10S) Bar graph of relative CSPG intensity in injured versus uninjured areas.

FIGS. 11A-11D show immunocytochemistry of biomarkers in microglia maturation culture. (FIG. 11A) Schematic illustration of EMPs and microglia induction. (FIG. 11B-FIG. 11E) Representative fluorescence micrographs of microglia marker (CX3CR1, TMEM119, IBA-1). (FIG. 11C and FIG. 11E are images at high magnification of the regions outlined by the red boxes in FIG. 11B and FIG. 11D, respectively). Scale bars: 20 μm.

FIGS. 12A-12H show microglia assembly pathway 2 (FIG. 12A) Schematic illustration of microglia assembly pathway 2. (FIG. 12B) Representative bright-field micrographs of mature microglia like cells. (FIG. 12C) Schematic illustration of a protocol for the assembly 2. Mature microglia were assembled in freshly injured organoids (24-week) in a medium supplemented with IL-34 and mCSF. Organoids in 96 wells were then agitated at 55 rpm with orbital shaking. (FIG. 12D) Fluorescence micrographs of CSPG (green) and IBA-1 (red) and DAPI, blue). (FIG. 12E) Representative fluorescence micrographs of IBAI (red) and GFAP (green). (FIG. 12F) Fluorescence micrographs of TUJ-1 (green) and DAPI (blue) in conditions with E2 or IKAVA PAs (DAPI, blue). (FIG. 12G) Bar graphs of relative TUJ-1 intensity compared to uninjured arca. (FIG. 12H) Fluorescence micrographs of neurofilament (yellow) specific for axon represented in injured organoid surface treated with E2 or IKAVA PAS (DAPI, blue). Scale bars: 200 μm.

FIGS. 13A-13C show transcriptomic analysis of injured and treated human spinal cord organoids (hSCOs). (FIG. 13A) Volcano plots illustrate differential gene expression in organoids treated with IKVAV PA relative to E2 PA (statistically significant changes in −log₁₀ p-values and expression magnitude by log₂ fold changes are indicated by red dots). (FIG. 13B) Top 50 differentially expressed genes identified by RNA sequencing of organoids treated with IKVAV PA and E2 PA (known inflammatory genes are marked by arrows). (FIG. 13C) Gene ontology analysis of the top 50 differentially expressed genes revealed enrichment of signatures of inflammation. GO:0050865, regulation of cell activation, R-HSA-6798695, Neutrophil degranulation: GO:002697, regulation of immune effector process; GO:0050778: positive regulation of immune response; GO:0045321: leukocyte activation; GO:0001819: positive regulation of cytokine production; WP3937: microglia pathogen phagocytosis pathway; GO:0050727: regulation of inflammatory response; R-HSA-1280218: Adaptive immune system; GO:0040017: positive regulation of locomotion; GO:0040017: positive regulation of locomotion;

WP3945: TYROBP causal network in microglia; GO:0071345: cellular response to cytokine stimulus; GO:0006935: chemotaxis; GO:0009617: response to bacterium; GO:0051046:regulation of secretion; GO:0007159: leukocyte cell-cell adhesion; GO:0098657: import into cell; GO:0002274: myeloid leukocyte activation; M5885: NABA MATRISOME ASSOCIATED; GO:0050777: negative regulation of immune response.

### DETAILED DESCRIPTION

Provided herein are engineered human spinal cord organoids, methods for generating the same, and methods of use thereof for development and testing of therapeutic treatment for spinal cord injury.

Regeneration upon injury is a milestone to understand the processes of organogenesis and its repair and to reveal cell plasticity and potency. Injury in organs triggers cellular and molecular rearrangements that induce intrinsic repair. However, the central nervous system in humans has poor regeneration capacity. Following spinal cord injury (SCI), specialized spinal cord communications that rely on neural cell networks, neurotransmitters, and electrical wiring, can be disrupted, leading to neurodegeneration and irreversible paralysis in humans. The current paradigm for understanding the pathophysiology and potential treatment of SCI relies largely on animal model studies. Yet, spinal cord injury therapeutics remain challenging due to a lack of experimental models that can recapitulate the injury environment, neuroinflammation, and associated glial scar in humans. The present disclosure addresses these limitations provides human spinal cord organoids, methods of development thereof, and methods of use thereof in modeling spinal cord injury and assessing potential therapeutic agents for treatments thereof.

Experiments conducted during development of embodiments herein demonstrate that human spinal cord organoids post mechanical injury develop localized glial scar-like tissues reminiscent of spinal cord injury observed in human injuries. Human GFAP+ glial cells created a border distinguishable from TUJ-1+ neurons in the following weeks post-injury. In striking contrast, a developmental SCI treatment composed of bioactive peptide nanofibers prevented the glial scar formation and promoted neurite extension at the injury site, indicating that the bioactive nanofibers provide structural support and induce neuronal axon repair in human spinal cord tissue. The findings provide unprecedented avenues to model human-specific responses to injury and repair, serving as a scalable platform for testing candidate therapeutic molecules.

Experiments conducted during development of embodiments herein demonstrate that human spinal cord organoids (hSCOs) directed by developmental signals enabled dissection of the processes of spinal cord formation, in particular, neurogenesis and gliogenesis with histological information. In combination with scRNAseq and bioinformatic analysis, experiments were conducted during development of embodiments herein to unbiasedly identify a variety of spinal cord cell types based on known spinal cord signature genes. Activity of the bioactive peptide IKVAV (SEQ ID NO: 1) on nanofiber scaffolds were shown herein to cause neuronal axon extension in an injury specific manner. In some embodiments, the engineered human spinal cord organoids provided herein have neuronal cells, astroglial cells, and immune cells, such as microglia.

Neuroinflammation involves acute and subacute injury phases that regulate scar formation that eventually leads to chronic injury. SCI in humans is characterized by a cavity filled with cerebrospinal fluid which is surrounded by scar containing various types of fibroblasts, astroglia, and immune cells. In this niche, growth of spared neurons surrounding the injury site is inhibited by both chemical signals and physical barriers. However, current research tools are insufficient to study neuroinflammation-dependent processes that regulate the injury phases in humans, thereby limiting the development of novel translatable therapies. Here, human spinal cord organoids that model neuroinflammation in the context of SCI were developed. These tissues are the first immune-competent human spinal cord injury organoids known to the present inventors, can be used as a tool to greatly accelerate discoveries of therapeutic interventions for SCI and also potentially for brain injury, stroke, and early detection of brain ischemia. Furthermore, the model described here is highly relevant to treatments of various degree of conditions, including acute and subacute SCI, and can also be used to search for therapies that address chronic injuries. Herein, neuroimmune human spinal cord organoids containing neurons, astroglial cells, and microglia were developed. These neuroimmune hSCOs are particularly useful in recapitulating the glial scar and deposition of chondroitin sulfate proteoglycans (CSPGs), effectively mimicking characteristics of injury in adult tissues.

The spinal cord organoid systems herein allow for the study of molecular and cellular mechanisms associated with human neurons, astroglia, and microglia. Mechanical injury of the hSCOs herein recapitulates localized glial scar-like tissue formation with a known injury marker in human species. Demonstrated efficacy of therapeutic treatment by peptide amphiphile nanofibers comprising the bioactive peptide IKVAV (SEQ ID NO: 1) that can prevent the glial scar and promote neurite extension, thus demonstrating the possible use of this system for testing and validating possible therapeutic treatments for spinal cord injury in humans.

### 1. Definitions

To facilitate an understanding of the present technology, a number of terms and phrases are defined below. Additional definitions are set forth throughout the detailed description.

The use of the terms “a” and “an” and “the” and “at least one” and similar referents in the context of describing the invention (especially in the context of the following claims) are to be construed to cover both the singular and the plural, unless otherwise indicated herein or clearly contradicted by context.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context.

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The terms “agent” and “drug” are used interchangeably herein in the broadest sense and refer to any substance that can be administered to a subject or is a potential candidate for administration to a subject, including as a therapeutic or a prophylactic.

As used herein, the term “glial cells” is understood to refer to macroglia, and is inclusive of astroglia (also referred to as “astrocytes”), oligodendrocytes (e.g. myelin-producing cells), and ependymal cells.

A “glial scar-like tissue” as used herein refers to a tissue formed in an hSCO after injury that resembles the glial scar that forms in vivo in the central nervous system of a subject following an injury in the subject. A glial scar-like tissue contains reactive astrocytes and extracellular matrix components and forms in response to an injury as a result of a process referred to as reactive astrogliosis.

The term “organoid” as used herein refers to a structure that mimics one or more characteristics of an organ. A “human spinal cord organoid” refers to a three-dimensional structure that mimics one or more characteristics of the human spinal cord. For example, a human spinal cord organoid (hSCO) may comprise one or more cell types and/or tissue types present in the human spinal cord. For example, a hSCO may comprise neural cells (e.g. neurons, neural stem cells), astroglial cells (i.e. astroglia also referred to as “astrocytes”), and immune cells (e.g. microglia). As another example, the structural organization of one or more cell/tissue types in a hSCO may be similar to the organization seen in a human spinal cord in vivo.

### 2. Human Spinal Cord Organoids and Related Methods

In some aspects, provided herein are human spinal cord organoids (hSCOs) comprising neural cells (e.g. neural stem cells, neurons such as motor neurons), astroglial cells (i.e. astrocytes also known as astroglia), and immune cells. In some embodiments, the immune cells comprise microglia, macrophages, neutrophils, T-cells, or a combination thereof. In some embodiments, the immune cells comprise microglia.

In some embodiments, the neural cells express class III beta-tubulin (TUJ-1). In some embodiments, the astroglial cells express GFAP. In some embodiments, the microglia express IBA-1. In some embodiments, neutrophils express CD14, CD13, CD15, CD16, CD33, CD62L, CXCR2, CXCR4, CD18, CD24, CD11b, CD11c, CD177, CD16, CD87, CD10, MPO, CD35, CD45, or CD49d. In some embodiments, T-cells comprise cytotoxic T-cells (e.g. expressing CD8) and/or helper T-cells (e.g. expressing CD4). In some embodiments, macrophages express CD11b, CD68, F4/80, CD14, or CCR5. Other suitable markers for the above cell types may be used to verify expression in the hSCOs.

In some embodiments, the hSCO further comprises oligodendrocytes (myelin-producing cells) and/or ependymal cells. For example, GFAP may serve as a marker for astrocytes and ependymal cells, and OLIG2 may serve as a marker for oligodendrocytes.

In some embodiments, the hSCO is substantially spherical in shape. In some embodiments, the hSCO is substantially spherical in shape and has an average diameter of at least 2 mm. In some embodiments, the hSCO is substantially spherical in shape and has an average diameter of about 2 mm to about 10 mm. In some embodiments, the hSCO is substantially spherical in shape and has an average diameter of about 2 mm to about 10 mm, about 2 mm to about 9.5 mm, about 2 mm to about 9 mm, about 2 mm to about 8.5 mm, about 2 mm to about 8 mm, about 2 mm to about 7.5 mm, about 2 mm to about 7 mm, about 2 mm to about 6.5 mm, about 2 mm to about 6 mm, about 2 mm to about 5.5 mm, about 2 mm to about 5 mm, about 2 mm to about 4.5 mm, about 2 mm to about 4 mm, or about 3 mm. In some embodiments, the hSCO is substantially spherical in shape and has an average diameter of at least 2.5 mm. In some embodiments, the hSCO is substantially spherical in shape and has an average diameter of at least 2.6 mm, 2.7 mm, 2.8 mm, 2.9 mm, or about 3 mm. In some embodiments, the hSCO is substantially spherical in shape and has an average diameter of about 3 mm.

In some embodiments, the hSCO comprises multiple layers of tissue. In some embodiments, the hSCO comprises one or more tissue layers comprising neurons, and one or more tissue layers comprising astroglial cells and microglia. For example, in some embodiments an inner portion of the hSCO (e.g. inner tissue layers of the hSCO) comprise neurons, and an outer layer/outer surface of the hSCO comprises astroglial cells and microglia. In some embodiments, the neurons are characterized by expression of the neuronal marker TUJ-1 and a neuronal morphology including a cell body/soma, axon, and dendrites. In some embodiments, the neurons (e.g. the neurons present on the inner layers) contain neurites. In some embodiments, astroglial cells and microglia are present on an outer surface of the organoid, and the outer surface is substantially smooth. In some embodiments, astroglial cells are characterized by expression of the glial cell marker GFAP and a glial cell morphology. In some embodiments, microglia are characterized by expression of the mature microglia marker IBA-1. In some embodiments, during the culture and developmental process of the hSCO, the outer surface of the organoid transforms from a topographically rough surface to a topographically smooth surface covered by astroglial cells and having a relatively flat morphology. In some embodiments, microglia are added to and infiltrate this outer surface.

In some embodiments, the hSCO has been cultured for at least about 8 weeks. In some embodiments, the hSCO has been cultured for at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least about 17 weeks, at least about 18 weeks, at least about 19 weeks, at least about 20 weeks, at least about 21 weeks, at least about 22 weeks, at least about 23 weeks, at least about 24 weeks, at least about 25 weeks, at least about 26 weeks, at least about 27 weeks, or at least about 28 weeks. In some embodiments, the hSCO has been cultured for about 8 weeks to about 28 weeks.

In some embodiments, the hSCO has received an injury and comprises a glial scar-like tissue and deposits of chondroitin sulfate proteoglycans (CSPGs) as a result of the spinal cord injury. CSPGs refer to proteoglycans including a protein core and a chondroitin sulfate side chain. CSPGs are structural components of a variety of human tissues and play roles in neural development and glial scar formation. CSPGs are implicated in inhibition of axonal regeneration and neurogeneration after central nervous system injury. In some embodiments, the CSPGs are a part of the glial scar-like tissue that forms after the injury. In some embodiments, the injury is a mechanical injury as a result of cutting or applying a compressive impact force, as described in more detail below.

The hSCOs described herein find use in methods of simulating spinal cord injury. Simulating spinal cord injury may provide useful information about injury-related pathways that occur during the spinal cord injury and therefore elucidate potential therapeutic targets to treat spinal cord injury. The hSCOs described herein further find use in methods of simulating spinal cord injury and assessing potential treatments for the injury.

In some aspects, provided herein are methods of simulating a spinal cord injury using the hSCOs provided herein. In some embodiments, provided herein are methods of simulating a spinal cord injury comprising injuring a human spinal cord organoid comprising neural cells and astroglial cells. In some embodiments, the hSCO comprises neural cells, astroglial cells, and immune cells. In some embodiments, the immune cells comprise microglia, macrophages, neutrophils, T-cells, or a combination thereof. In some embodiments, provided herein are methods of simulating a spinal cord injury comprising injuring a human spinal cord organoid comprising neural cells, astroglial cells, and microglia.

In some embodiments, the hSCO comprises neural cells and astroglial cells, and immune cells or precursors thereto (e.g. immune cell progenitors) are added to and infiltrate the hSCO after injury. In some embodiments, the immune cells comprise microglia, macrophages, neutrophils, T-cells, or a combination thereof. In some embodiments, immune cell progenitors are added to the hSCO and mature in the hSCO. For example, in some embodiments microglia are added to and infiltrate the hSCO after injury. For example, in some embodiments microglia are added to the hSCO and infiltrate areas where astroglial cells (e.g. GFAP positive cells) are present in the hSCO.

In some embodiments, injuring the human spinal cord organoid comprises mechanically injuring the hSCO. In some embodiments, mechanically injuring the hSCO comprises cutting the hSCO or applying a compressive impact force to the hSCO. In some embodiments, cutting the hSCO comprises forming an incision of a suitable depth and length using a tool, such as a blade or a scalpel. In some embodiments, mechanically inuring the hSCO comprises applying a compressive impact force to the hSCO. The strength and duration of the compressive impact force can be selected to achieve any desired degree of damage to the hSCO. For example, the hSCO may be used to simulate a mild spinal cord injury by applying a relatively mild compressive impact force to the hSCO, a moderate spinal injury by applying a moderate compressive impact force to the hSCO, or a severe spinal cord injury by applying a severe compressive impact force to the hSCO. For example, in some embodiments mechanically injuring the hSCO comprises applying a compressive impact force of about 20 kilodynes (kdyn) to about 300 kilodynes of force to the hSCO. For example, in some embodiments mechanically injuring the hSCO comprises applying a compressive impact force of about 20 kdyn to about 300 kdyn, about 20 kdyn to about 250 kdyn, about 20 kdyn to about 200 kdyn, about 20 kdyn to about 180 kdyn, about 30 kdyn to about 170 kdyn, about 40 kdyn to about 160 kdyn, about 50 kdyn to about 150 kdyn, about 60 kdyn to about 140 kdyn, about 70 kdyn to about 130 kdyn, or about 80 kdyn to about 120 kdyn of force. In some embodiments, the dwell time of the force is about 1 second to about 10 seconds (e.g. 1 second, 2 seconds, 3 seconds, 4 seconds, 5 seconds, 6 seconds, 7 seconds, 8 seconds, 9 seconds, 10 seconds).

In some embodiments, the method of simulating a spinal cord injury further comprises assessing a potential therapeutic agent for the treatment of the spinal cord injury. In some embodiments, provided herein are methods of assessing a potential therapeutic agent for treatment of spinal cord injury comprising contacting an injured hSCO with the potential therapeutic agent and assessing a response in the hSCO. In some embodiments, provided herein are methods of assessing a potential therapeutic agent for treatment of spinal cord injury, comprising obtaining a human spinal cord organoid described herein (e.g. an hSCO comprising neural cells and glial cells), injuring the hSCO (e.g. by cutting or applying a compressive impact force), and contacting the injured hSCO with a potential therapeutic agent for the treatment of spinal cord injury. In some embodiments, the methods provided herein comprise contacting the injured hSCO with a potential therapeutic agent for the treatment of spinal cord injury, and assessing a response of the injured hSCO to the potential therapeutic agent. In some embodiments, assessing a response of the injured hSCO to the potential therapeutic agent comprises obtaining a baseline indication of injury in the hSCO, and evaluating whether the baseline indication of injury improves after contacting the injured hSCO with the potential therapeutic agent. An improvement indicates that the agent may be an effective therapy for treatment of spinal cord injury in subjects, including in human subjects. In some embodiments, a baseline indication of injury is obtained by evaluating/quantifying formation of a glial scar (e.g. a scar containing reactive astrocytes and extracellular matrix components) and/or deposition of chondroitin sulfate proteoglycans (CSPGs) on the organoid. In some embodiments, injuring the hSCO produces a glial scar-like tissue in the organoid. In some embodiments, the potential therapeutic agents reduces the glial scar/glial scar-like tissue and/or reduces deposition of CSPGs in the hSCO. For example, in some embodiments the potential therapeutic agent reduces the size of the glial scar/glial scar-like tissue, or prevents significant formation of a glial scar/glial scar-like tissue. As another example, in some embodiments the potential therapeutic agent reduces deposition of CSPGs on the organoid. In some embodiments, a baseline indication of injury is obtained by evaluating neuronal cell death or damage following injury. In some embodiments, the potential therapeutic reduces the amount of neuronal cell death following injury and/or promotes neurogenesis following injury. In some embodiments, the potential therapeutic agent promotes neurite outgrowth from damaged neurons following injury.

In some aspects, provided herein are methods of generating human spinal cord organoids. In some embodiments, provided herein is method of generating a human spinal cord organoid (hSCO) comprising neural cells, astroglial cells, and microglia. In some embodiments, provided herein is a method of generating a human spinal cord organoid (hSCO) comprising neural cells, glial cells, and immune cells, comprising: generating hSCOs comprising neural cells and astroglial cells, co-culturing the hSCO with precursors of immune cells, and inducing maturation of the precursor cells into immune cells. In some embodiments, the precursors of immune cells comprise precursors of microglia, macrophages, neutrophils, T-cells, or a combination thereof.

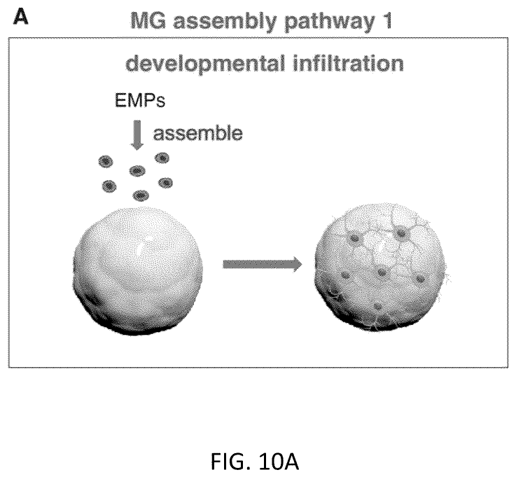

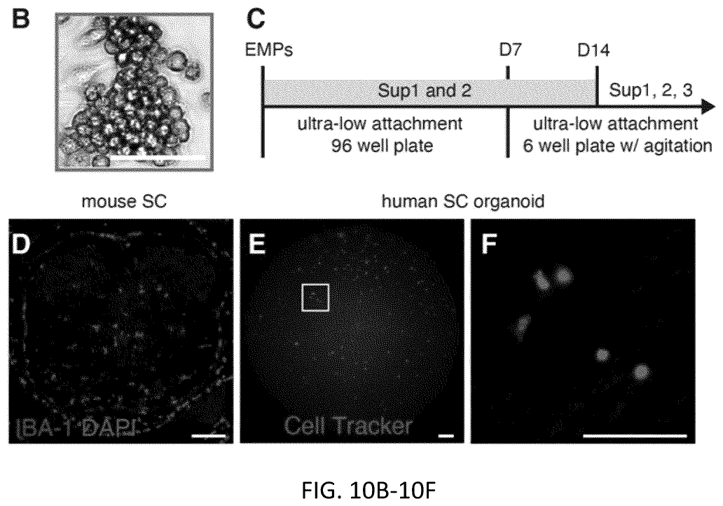

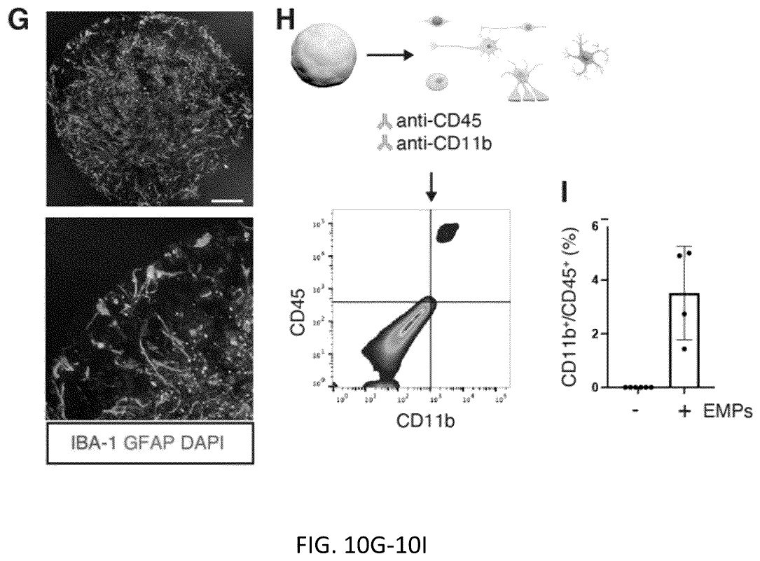

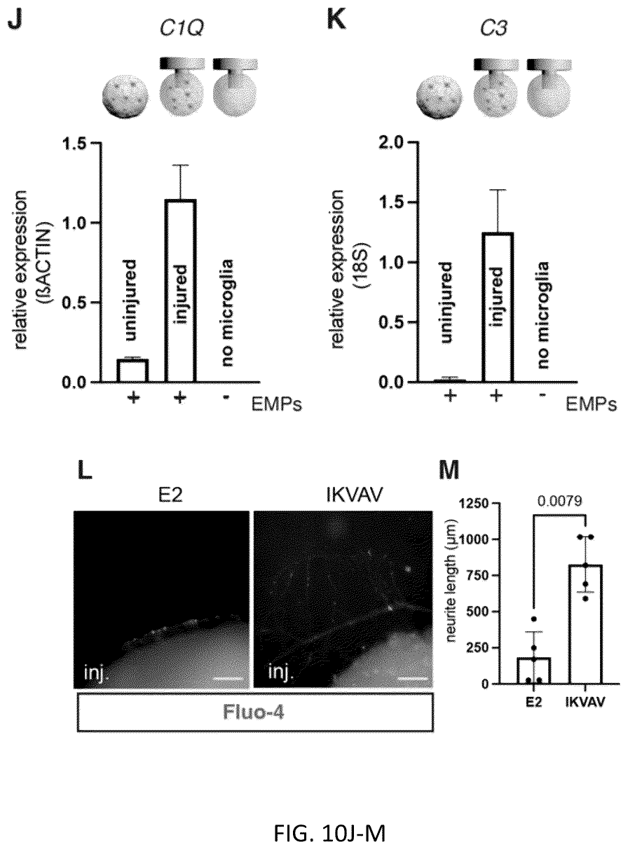

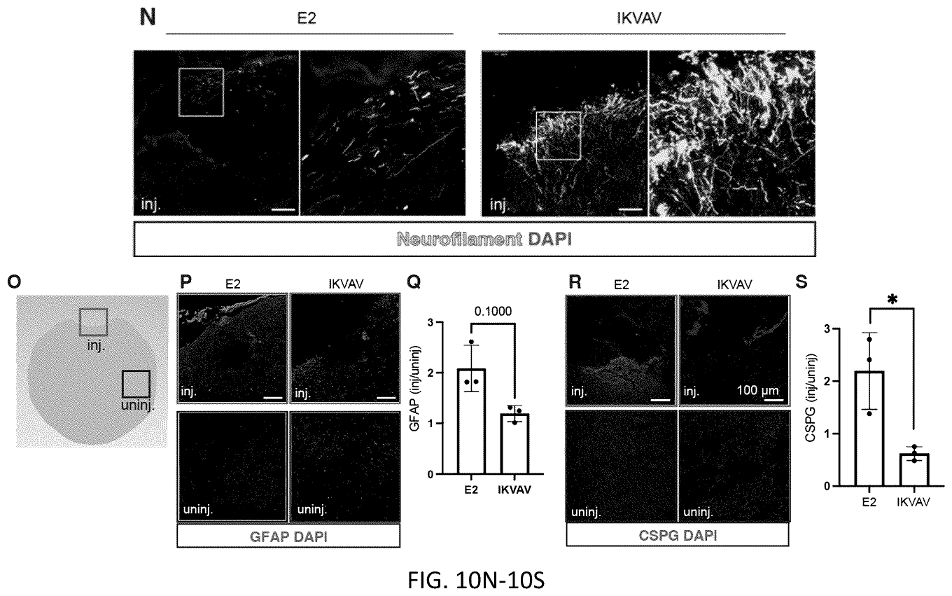

In some embodiments, the method of generating hSCOs comprises culturing human induced pluripotent stem cells (hiPSCs) in a series of spinal cord induction mediums, thereby inducing formation of spinal cord progenitor organoids (SPOs), followed by culturing the SPOs in spinal cord organoid maturation medium (SCMM). In some embodiments, the method of generating hSCOs comprises culturing human induced pluripotent stem cells (hiPSCs) in a series of spinal cord induction media, thereby inducing formation of spinal cord progenitor organoids (SPOs), followed by culturing the SPOs in spinal cord organoid maturation medium (SCMM) with orbital shaking for a period of at least 6 weeks (e.g. at least 6, weeks, at least 7 weeks, at least 8 weeks, at least 9 weeks, at least 10 weeks, at least 11 weeks, at least 12 weeks), thereby generating hSCOs comprising neural cells and astroglial cells. In some embodiments, the method further comprises co-culturing the 7-week hSCO with erythro-myeloid progenitor cells (EMPs), and first inducing differentiation of the EMPs into immature microglia and then guides them to become mature microglia (see drawings FIG. 10C for two step procedures in EMP co-culture by sup 1 and 2 for differentiation until day 14 and then sup1, 2, 3 for maturation taking 1-2 weeks). In some embodiments, inducing maturation of the EMPs comprises culturing the hSCO—EMP co-culture with a microglia maturation medium comprising one or more factors that promotes maturation of microglia. In some embodiments, culture in the microglia maturation medium takes 1-2 weeks for microglia to mature.

In some embodiments, culturing hiPSCs in the series of spinal cord induction mediums comprises culturing the hiPSCs in a first spinal cord induction medium comprising initial instructional growth factors, followed by culturing the hiPSCs in a second spinal cord induction medium comprising secondary instructional growth factors, followed by culturing the hiPSCs in a third spinal cord induction medium comprising retinoic acid, thereby inducing formation of SPOs. In some embodiments, culturing hiPSCs in the series of spinal cord induction media comprises culturing the hiPSCs in a first spinal cord induction medium comprising initial instructional growth factors for 24 hours to 6 days, followed by culturing the hiPSCs in a second spinal cord induction medium comprising secondary instructional growth factors for 24 hours to 6 days, followed by culturing the hiPSCs in a third spinal cord induction medium comprising retinoic acid, thereby inducing formation of SPOs. In some embodiments, culturing hiPSCs in the series of spinal cord induction media comprises culturing the hiPSCs in a first spinal cord induction medium comprising initial instructional growth factors for about 3 days, followed by culturing the hiPSCs in a second spinal cord induction medium comprising secondary instructional growth factors for about 3 days, followed by culturing the hiPSCs in a third spinal cord induction medium comprising retinoic acid, thereby inducing formation of SPOs. In some embodiments, the initial instructional growth factors comprise recombinant human basic fibroblast growth factor (FGF), a glycogen synthesis kinase 3 (GSK3) inhibitor, and a TGFβ receptor inhibitor. Exemplary GSK3 inhibitors include SB-216763, CHIR99021, BIO (6-bromoindirubin-3′-oximc), and LY2090314. In some embodiments, the GSK3 inhibitor is CHIR99021. In some embodiments, the TGFβ receptor inhibitor is a type I receptor kinase (ALK5) inhibitor. Exemplary ALK5 inhibitors include A77-01, A83-01, AZ 12799734, D4476, GW788388, IN1130, LY2109761, R268712, SB431542, SB505124, SB525334, SD208, and SM16. In some embodiments, the ALK5 inhibitor is SB431542. In some embodiments, the secondary instructional growth factors comprise an ALK5 inhibitor and retinoic acid. For example, in some embodiments the secondary instructional growth factors comprise SB431542 and retinoic acid. In some embodiments, the third spinal cord induction medium comprises retinoic acid and does not comprise the initial instructional growth factors (e.g. does not comprise FGF, the GSK3 inhibitor, or the TGFβ receptor inhibitor). In some embodiments, the third spinal cord induction medium comprises retinoic acid and does not comprise any of FGF, a GSK3 inhibitor, or a TGFβ receptor inhibitor. In some embodiments, the method comprises culturing the hiPSCs in the third spinal cord induction medium for 5-15 days (e.g. 5-15 days, 6-14 days, 7-13 days, 8-12 days, 9-11 days, or about 10 days). In some embodiments, the method comprises culturing the hiPSCs in the third spinal cord induction medium for about 10 days.

In some embodiments, the spinal cord organoid maturation medium comprises recombinant human BDNF, recombinant human GDNF, and retinoic acid. In some embodiments, method comprises culturing the SPOs in spinal cord organoid maturation medium with shaking on an orbital shaker (e.g. with orbital shaking) for at least 6 weeks (e.g. at least 6 weeks, at least 7 weeks, at least 8 weeks, at least 9 weeks, at least 10 weeks, at least 11 weeks, at least 12 weeks. In some embodiments, the method comprises culturing the SPOs in spinal cord organoid maturation medium with orbital shaking for at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, or more than 6 months.

### EXPERIMENTAL

### Example 1

### Materials and Methods

The following exemplary materials and methods were used in the experiments conducted herein.

Human-Induced Pluripotent Stem Cell (hiPSCs) Maintenance

The hiPSCs 18a were cultured on Matrigel (Corning #356230)-coated dishes with mTeSR1 (Stem Cell Technologies, Catalog #85850). The cells were dissociated to single cells with 1 mM EDTA or Accumax (Innovative Cell Technologies, Catalog #AM105-500) for passaging or differentiation, respectively. Matrigel lots were routinely tested to optimize stem cell maintenance. ROCK Inhibitor, Y-27632 dihydrochloride (Tocris Bioscience, Catalog #1254) was used to prevent cell death upon cell dissociation and removed from the culture after stem cell colony formation.

Human Spinal Cord Organoid (hSCO) Generation

hSCO were generated from hiPSCs. hiPSC colonies were seeded at the density of 10K cells per well into PrimeSurface® 3D Culture Spheroid plates (Sumitomo Bakelite Co., Ltd. #MS-9096UZ) in a spinal cord induction medium (SCIM) containing DMEM/f12, neurobasal-A (Life Technologies, 10888022), 10% KSR, N2 supplement, B-27 supplement without vitamin A (Life Technologies, 12587010), GlutaMAX (1:100, Life Technologies), penicillin and streptomycin (1:100, Life Technologies) and supplemented with 10 μM Y-27632 dihydrochloride, 20 ng ml⁻¹ recombinant human basic FGF (Pepro Tech Inc, #10018B), 3 μM CHIR99021 (StemGent, #04-0004-base) and 10 μM SB431542 (Tocris, #1614) from day 0 to day 3. The day on which the organoid production starts is defined as day 0. From day 3 to day 6, the organoids were cultured in SCIM supplemented with 10 μM SB431542, 100 nM retinoic acid (Sigma-Aldrich, #R2625) and 0.5 μM SAG (Enzo Life Sciences, Inc, #ALX-270-426-M001). Next from day 6 to day 15, half the medium was performed with SCIM supplemented with 100 nM retinoic acid and 0.5 M SAG. On the day 15 culture, the growing organoids were transferred into ultra-low adhesion 6 well dishes (Corning, #3471) in spinal cord maturation medium (SCMM) containing N2B27 medium supplemented with 1 mM L-GlutaMAX, 0.1 mM 2-mercaptoethanol (Sigma-Aldrich, #63689-25ML-F), 0.5 μM L-ascorbic acid (Sigma-Aldrich, #A4544), 10 ng ml⁻¹ recombinant human BDNF (Pepro Tech Inc, #450-02-10UG), 20 ng ml⁻¹ recombinant human GDNF (Pepro Tech Inc, #450-10-10UG) and 100 nM retinoic acid. The plates were placed in an incubator-docked active arbitrary shaker system to agitate the culture (Orbi-Shaker CO2 Remote Control Shaker, Fisher Scientific, #50-197-6821). Culture agitation on an orbit shaker in a tissue culture incubator is critical to allow for homogenous culture conditions and to protect human spinal cord organoids from accumulation of local waste products. Half-medium change was performed every three days.

### Scalpel Injury in Human Spinal Cord Organoids

Mechanical injuries were induced in the organoids between 12 and 24 weeks in culture. To initiate hemisection injury, the organoids are cut with a sterilized scalpel blade (World Precision Instruments, #500348) on the 10 cm petri dish. Injured organoids were transferred into 24 well plates to be cultured for a month with half medium change with the spinal cord medium containing N2B27 medium supplemented with 1 mM L-Glu, 0.1 mM 2-ME, 0.5 μM ascorbic acid, 10 ng ml⁻¹ recombinant human BDNF, 20 ng ml⁻¹ recombinant human GDNF and 100 nM retinoic acid. The media was sampled for use in an LDH assay to monitor cytotoxicity by the scalpel injury relative to the media prior to the injury. In four weeks, tissues were fixed in 4% PFA/PBS followed by immunohistological analysis and microscopic imaging.

### Contusion Injury of Spinal Cord Organoid

A severe contusive injury was performed on the organoid using the Infinite Horizon Spinal Cord Impactor system (IH-0400 Precision Systems and Instrumentation) with 85 kdyn of impact force and a dwell time of 1˜5 seconds under the Force mode in IH Spinal Cord Impactor v5.0. software. A probe with a diameter of 1 mm was used. After the damage, the organoids were transferred to a culture dish filled with media and cultured for 24 hours. The media was sampled and an LDH assay was performed to determine the cytotoxicity post-injury relative to the media prior to the injury. These organoids were cultured for four weeks with exchange of the medium every three days.

### Peptide Amphiphile (PA) Nanofiber Preparation

PAs were synthesized in the Peptide Core Facility at Northwestern University using solid-phase peptide synthesis and purification by high-performance liquid chromatography. The sequence of synthesized PAs are as follows: PAI: C₁₆-VVAAEEEEGIKVAV (SEQ ID NO: 2), PA2: C₁₆-AAGGEEEEGIKVAV (SEQ ID NO: 3) and control E2 PA: C₁₆-VVAAEE (SEQ ID NO: 4)). After lyophilization of the purified PAs, the PA powder was reconstituted to a concentration of 1 mM in sterile isotonic saline sodium chloride, 9.9% (w/v) (Ricca Chemical) solution and adjusted to a pH of 7.4 using additions of 0.2 M NaOH to ensure cell compatibility and material consistency. Bath sonication (Branson CPX1800H Digital Heated Ultrasonic Cleaner) was performed for 20 min. PA solutions were then annealed at 80° C. for 30 min and then slowly cooled to 25° C. overnight. Alternatively, for small scale such as 100 μL per tube, a thermal cycler (Eppendorf PCR Thermocycler) was used for even and controlled heating and cooling of all samples with a program of a “hot start” at 80° C. and cooling to 25° C. at a rate of 1° C./min. When fluorescent dyes were needed for imaging, labeled and unlabeled PA powders were separately dissolved in sterile isotonic saline sodium chloride, 09% (w/v) (Ricca chemical) and were then pH adjusted to 7.4 using 1 M NaOH. The solutions were then mixed to give 99 mol % non-labeled PA and 1 mol % Alexa 647 labeled PA. Samples were briefly vortexed and then bath-sonicated for 20 min. The mixture was annealed at 80° C. for 30 min and slowly cooled to room temperature.

### PA Treatment in Injured Spinal Cord Organoid Models

For hemisection injury, E2 or IKVAV (SEQ ID NO: 1) PA nanofibers (e.g. PA1, PA2) were dispensed by a sharp tip to the legion sites a day post-SCI. Half the medium was exchanged every three days for a month culture. Neurites were visualized by neuronal cell-compatible dye Fluo-4 (ThermoFisher, F14201). After the Fluo-4 imaging, the organoids were fixed in 4% PFA/PBS followed by immunohistology analysis. For contusion injury, the PA nanofibers were injected 24 h after SCI using a glass capillary micropipette (Sutter Instruments, Novato, CA) (outer diameter, 100 μm). The capillaries were loaded onto a Hamilton syringe using a female Luer adaptor (World Precision Instruments) controlled by a Micro4 microsyringe pump controller (World Precision Instruments). The tip of the syringe was sticked the injured surface of the organoid, and 5-10 μL of the diluted amphiphile solution was injected at 1 μL/min. At the end of the injection, the pipette was left in place for an additional 2-3 min to allow material gelation followed by culture in the SCMM, the medium described above.

### Immunohistochemistry and Imaging

Mouse embryo isolation was performed. Time-pregnant CD1 mice (Charles River Laboratories) was sacrificed by cervical dislocation and the embryos were extracted at embryonic day 10, 14 or 30 days after birth. Spinal cords were dissected in PBS and fixed in 4% paraformaldehyde in 0.1 M PBS (pH 7.4). After overnight protection in 30% sucrose/PBS, tissues were embedded in sucrose/OCT (Tissue-Tek) and sectioned in a cryostat. The following primary antibodies were used: anti-NESTIN (1:1000, Sigma-Aldrich, #ABD69), anti-TUJ-1 (1:1000, Covance catalog #PRB-435P), GFAP (1:1000, DAKO, Z0334), OLIG2 (1:250, Millipore, #AB9610) and LAMININ (1:250, Sigma-Aldrich, #L9393). IBAI (rabbit, 1:500, ThermoFisher scientific, NC9288364: guinea pig, 1:500, Synaptic Systems, 234 308: goat, 1:500, R&D Systems, MAB7308SP: goat, 1:500, Abcam, ab5076). TMEM119 (1:1000, Abcam, ab 185333), MBP (rabbit 1:1000, Abcam, ab40390: mouse, 1:1000, CST, 83683), MAP2 (1:1000, BioLegend, #822501), Neurofilament marker (SMI312) (mouse 1:2000, BioLegend, 837904). The following secondary antibodies were used: Alexa 488-conjugated goat anti-chicken (1:1000, A11039, Invitrogen). Alexa 488-conjugated donkey anti-rabbit (1:1000, A-21206, Invitrogen), Alexa 488-conjugated donkey anti-goat (1:1000, A-11055, Invitrogen), Cy3-conjugated donkey anti-rabbit (1:200, 711-165-152, Jackson ImmunoResearch), Cy3-conjugated donkey anti-mouse (1:200, 715-165-151, Jackson ImmunoResearch), Cy3-conjugated donkey anti-goat (1:200, 705-165-147, Jackson ImmunoResearch), Cy5-conjugated donkey anti-goat (1:200, 705-495-147, Jackson ImmunoResearch), Jackson ImmunoResearch), Cy5-conjugated donkey anti-rabbit (1:200, 711-495-152, Jackson ImmunoResearch). CY5-conjugated goat anti-Guinea Pig (1:200, Jackson 106-175-003, Jackson ImmunoResearch). For CS-56 (mouse IgM, 1:500, Sigma-Aldrich, C8035), corresponding secondary antibodies for IgM were used as follows: Alexa Fluor 568 goat anti-mouse IgM μ chain (1:500, Thermo Fisher Scientific, A21043). Alexa Fluor 647-AffiniPure Goat Anti-Mouse IgM u Chain Specific (1:500, Jackson Immuno Research Labs, 501-942-393). DAPI (1:1000) was used for DNA. For whole mount immunostaining, Triton X100 and Matrigel were additionally used followed by imaging by confocal microscope. AXR Confocal Microscope or Ts2-FL fluorescent microscopes were used for cryosectioned slide staining. AXR Confocal Microscope or Leica SP8 DiveB Multiphoton microscope were used for whole-mount immunostaining; Analysis Software: LAS X Office 5.1.0. Neurofilament quantification: The area of neurofilament staining was quantified using 3D Rendering in Imaris software (Bitplane, version 8.1). The cell surface function was applied to define the neurofilament area within the organoid.

### Human Spinal Cord Single-Cell RNA Sequencing

For preparation of single cells, the organoids were minced and collected in a six-well dish. Then the tissue was washed with PBS twice and 0.75 mL of warmed Papain (Worthington Biochemical Corporation) was added with DNase I (1:1000, Sigma). After 45 min, the tissue was carefully pipetted, and a stop solution was added. The dissociated solution was filtered with a 100 μm cell strainer (Falcon; Corning) and centrifuged at 1000 rpm at room temperature (RT) for 5 min. 1 mL HBSS (0.2% BSA fraction V) was added to the cell pellets and then filtered using a 40 μm cell strainer (Falcon; Corning). Cells were counted using a Cellometer K2 (Nexcelom) with acridine orange to calculate total number of nucleated cells and propidium iodide to count dead cells (cell viability exceeded more than 90%). The library preparation and sequence were performed at NUSeq Core of Center for Genetic Medicine. The single-cell organoid samples from three biologically independent experiments were pooled prior to the library generation. Single-cell 3′ RNA sequencing libraries were prepared using Chromium Single Cell v2 Reagent Kit and Controller (10X Genomics, Pleasanton, CA, USA). Libraries were assessed for quality (TapeStation 4200; Agilent, Santa Clara, CA, USA) and then sequenced on an HiSeq 4000 instrument (Illumina, San Diego, CA, USA). Raw reads were demultiplexed, filtered, and mapped to the human reference genome (hg38) using Cell Ranger 7.0.0 with default settings. The raw feature-barcode matrices generated by Cell Ranger were used for downstream analysis using Seurat v5.2.1. To exclude low-quality cells, standard filters were applied, including cells with higher than 7500 genes, cells with more than 15% mitochondrial genes, cells with fewer than 35% ribosomal genes, and cells with higher than 0.9% hemoglobin genes. The filtered expression data was normalized for each cell by the total expression, multiplied by a scale factor of 10,000, and then log-transformed. The cell-cycle phase scores were calculated for regression. The data was scaled using SCTransform (cell cycle results S.Score and G2M.Score, percentage of mitochondrial genes, number of features, and sample identities were regressed) to remove batch effects. Dimensionality reduction was done using Principal Component Analysis (PCA) and based on the results. PCs were used to identify clusters. The clusters were identified using the Approximate Nearest Neighbors method to identify a different resolution, 0.2, which was used for further downstream analysis and 12 clusters were identified. Marker genes were identified using FindAllMarkers (MAST method).

### Scanning Electron Microscopy (SEM)

Organoids were initially fixed in 4% PFA/PBS overnight. To allow imaging of the cross-sections, the organoids were then cut with a fresh scalpel and transferred to a second fixative solution containing 2% glutaraldehyde in 0.1 M sodium cacodylate buffer at 4° C. overnight and postfixed using 1% OsO4 in water for 2 hours. The specimens were then dehydrated in a graded series of ethanol (50%, 70%, 90%, and 100%), critical-point-dried with carbon dioxide (Samdri-790, Tousimis, Rockville, USA), mounted, and coated with 10 nm gold in a sputter coater. Finally, the specimens were observed under a scanning electron microscope (JCM-6000PLUS, JEOL, Japan).

### Whole-Mount Cell Viability and Cell Death Imaging

Live/dead analysis was performed one hour post-SCI via Cyto3D® Live-Dead Assay Kit. SCI organoids were transferred to an Ibidi thin layer plastic bottom dish or Matsunami glass bottom dish for fluorescent imaging in a wide-field microscope, ECLIPSE Ts2FL (Nikon, Inc.).

### Cytotoxicity Measurement Post-SCI

Cell viability was measured using CyQUANTTM LDH Cytotoxicity Assay (ThermoFisher, #20300), a colorimetric assay that quantitatively measures the catalytic activity of lactate dehydrogenase (LDH), an enzyme that is released upon cell lysis post cytotoxic treatments. The media was collected before or 24 hours after injury to measure the extracellular levels of LDH enzyme activity read by 490 nm or 680 μm absorbance in Cytatition3 imaging reader (BioTek instruments, Inc.). Excel and GraphPad Prism were used for further analyses. At least three biological independent experimental repeats with two replicates per condition.

### Polar Histogram Analysis

The measured angles of the neurite segment relative to the normal direction of the organoid surface at the beginning of the neurite were categorized into 18 intervals with 10 degree per step (−90° to 90)° and plotted as a polar histogram.

### Statistics & Reproducibility

All experiments were performed from a minimum of three independent repeat experiments. No data were excluded from the analyses. Representative micrographs are shown as similar results were obtained from the independent repeat experiments. Values are expressed as mean±standard error of the mean (S.E.M). Results were analyzed using GraphPad (GraphPad version 9.5.1) and Microsoft Excel. For statistical significance test, Student t-test and one-way ANOVA were used for two-group and multiple-group comparison, respectively. In addition, post-hoc Tukey's (all groups) or Dunnett's test (versus control) was applied to compare multiple groups. Differences with p-value<0.05 were considered statistically significant. *, ** or *** indicates a p-value is less than 0.05, 0.01 or 0.001, respectively. ns=non-significance.

### RNA Sequencing

Organoids were harvested (two biological independent repeat experiments) and total RNA was isolated using RNeasy Micro Kit (QIAGEN #74104). Prior to the library preparation, RNA quantification was performed by Qubit (Thermo Fisher). Next, a high sensitivity RNA assay was used to determine the quality of total RNA (bioanalyzer, Agilent Technologies). TruSeq mRNA-Seq Library Prep was performed, and the libraries were sequenced by Illumina HiSeq 4000 (50 bp, Single-Read, 300 million Reads). The quality of reads, in FASTQ format, was evaluated using FastQC. Reads were trimmed to remove Illumina adapters from the 3′ ends using cutadapt (doi.org/10.14806/ej.17.1.200). Trimmed reads were aligned to the human genome (hg38) using STAR. Read counts for each gene were calculated using htseq-count in conjunction with a gene annotation file for hg38 obtained from Ensembl (useast.ensembl.org/index.html). Normalization and differential expression were calculated using DESeq2 that employs the Wald test. The cutoff for determining significantly differentially expressed genes was an FDR-adjusted p-value less than 0.05 using the Benjamini-Hochberg method. The differentially expressed genes are generated as heatmaps and volcano plots created with R (version 3.3.1). A pathway analysis was performed using Metascape [metascape.org] to identify significant pathways among the significantly differently expressed genes.

### Microglia Induction

Erythro-myeloid progenitors (EMPs) were generated as described in the manufacturer's instructions for STEMdiff™ Hematopoietic Kit (STEMCELL Technologies, #05310). Briefly, the stem cell colonies by hiPSCs 18a were gently scraped and the cell clumps were counted. The clumps were transferred to a single 6 well dish coated with Matrigel (Corning, #356230) with mTeSRI (STEMCELL Technologies, #85850). After 24 hours, the medium was switched to erythromyeloid induction A in the kit for first 3 days and then to erythro-myeloid induction B for 11 days. Both erythromyeloid induction A and B media were changed half every other day. After a total of 2 weeks in the induction culture, EMP like cells in supernatant were collected by centrifugation 1,500 rpm for 5 min and live cells were counted by trypan blue staining for subsequent microglia cell assembly 1.

### Microglia Assembly Pathway 1

75,000 EMPs generated above were cocultured with one 7-week spinal cord organoid in PrimeSurface® 3D Culture Spheroid plates (Sumitomo Bakelite Co., Ltd. #MS-9096UZ) with the EMP organoid basal medium consisting of 50% of STEMdiff™ Microglia basal medium (Stem cell technologies #100-0019) and 50% of SCMM. The EMP organoid basal medium is then supplemented with 1× Insulin-Transferrin-Selenium-Ethanolamine (Gibco, #51500056), STEMdiff™ Microglia supplement 1 and 2. The plates were incubated with agitation 55 rpm by a CO2 resistant shaker (Benchmark Scientific, Z763438) for one week with half medium change every other day. The EMP organoids were transferred to ultra-low attachment surface plate (Corning, #3473) and cultured for 2 weeks. For microglia maturation, the medium was changed to SCMM, the medium described above supplemented with Microglia Maturation supplements 1, 2, 3 (Stem cell technologies #100-0020) and 1X Insulin-Transferrin-Selenium-Ethanolamine (Gibco, #51500056). The organoids were cultured with medium change every 3 days.

### Microglia Assembly Pathway 2

The EMPs above were cultured for microglia differentiation by the manufacturer's instructions for STEMdiff™ Microglia Differentiation Kit (Stem cell technologies #100-0019). 5×106 cells were seeded in a single well of the 6 well dish and cultured for 3 weeks with half medium change in every 3 days. In a following week, the medium was exchanged into Microglia Maturation kit (Stem cell technologies #100-0020) and the microglia progenitors were cultured at least for a week to obtain mature microglia before injury. To mimic the microglia in SCI model animal ranging from 10˜20%, 80,000 mature microglia were added to 24-week organoids injured by impactor in the SCMM supplemented with IL-34 (100 ng/ml: R&D system #5265IL010), mCSF (25 ng/ml: R&D system #21-6MC0-10) and 1×Insulin-Transferrin-Selenium-Ethanolamine (Gibco, #51500056) factors important for microglial survival, for 48 hours and transferred to a 24 well dish, ultra-low attachment surface plate (Corning, #3473).

### Microglia Progenitor Cell Tracking

EMPs were labeled with CellTracker Green CMFDA (ThermoFisher #C7025) for 3 days of co-culture. Dye was washed out with PBS and organoids were mounted on glass bottom dish (ibidi, #81156) for fluorescence imaging.

Flow Cytometry analysis

Organoids were washed with PBS and dissociated by papain enzyme based on the manufacturer's instructions (Worthington). Briefly, the tissues are transferred into 24 well ultra-low attachment surface plate (Corning, #3473) with papain solution for incubation at 37° C. with constant agitation for an hour and with a trituration by pipetting. 100 μm filtration were used to filter undissociated tissue fragments. The number of organoid derived viable cells were measured by a blood count chamber and trypan blue staining to prepare 1×10⁶ cells/100 μL PBS in 1.5 mL. The cells were stained by Zombie Violet (1:400, bioledgend #77477) on ice for 20 min to exclude dead cells during flow cytometer analysis. 1 mL of staining buffer (Biolegend #420201) was added and the cells were centrifuged at 300 g for 5 min. Immunostaining blocking solution consisting of 5 μL of TruStain FcX blocking (Biolegend #422302) and 90 μL of staining buffer for 10 min incubation at room temperature. The following antibodies were added and incubated on ice for 20 min to detect cell surface antigens: Alexa Fluor® 488 anti-human CD11b antibody (1:20, Bioledgend #301317), APC Anti-Human CD45 (HI30) (1:20, Tonbo Bioscience #50-0459-T100). Cells were analyzed by flow cytometry (Melody, BD). Negative staining was used for signal threshold for each channel.

### Quantitative PCR

Organoids were harvested post injury day 3 and total RNA was isolated using RNeasy Micro Kit (QIAGEN #74104). cDNA was generated and 20 ng was used for qRT-PCR amplification using SYBR green (Thermo Fisher Scientific, #4309155) in 96 well plate (Thermo Fisher Scientific, #4346907) with adhesive film (Thermo Fisher Scientific, #4313663). Primer sequences used in this study: human β-ACTIN, forward, 5′CATGTACGTTGCTATCCAGGC-3′ (SEQ ID NO: 5); reverse 5′-CTCCTTAATGTCACGCACGAT-3′ (SEQ ID NO: 6); human CIQA forward 5′-TCTGCACTGTACCCGGCTA-3′ (SEQ ID NO: 7); reverse, 5′-CCCTGGTAAATGTGACCCTTTT-3′ (SEQ ID NO: 8). For TaqMan™ Real-Time PCR, the reaction mix was prepared by mixing TaqMan Fast Advanced

Master Mix (2X) (Thermo Fisher #4444556) with the TaqMan Assay probe (20X), nuclease-free
water, and cDNA template. TaqMan Gene Expression Assays for human C3(Hs00163811_ml) were loaded into MicroAmp Fast 96-Well plates in triplicate and gene expression was measured on the CFX Connect Real-Time PCR System. Amplification parameters were set as follows: 95° C. for 20 seconds, followed by 45 cycles of 95° C. for 1 second and 60° C. for 20 seconds. Relative gene expression was determined by calculating the ΔCt values between C3 and the housekeeping gene of human 18S (Hs03003631_g1). Human β-ACTIN or 18S was used as a housekeeping gene (i.e., its expression was used to normalize gene expression levels). The relative level of each gene was calculated using the 2-ΔΔCt method and reported as relative fold-change mean +S.E.M.

### Example 2

### Human Spinal Cord Organoids

Damage of the spinal cord, which can lead to irreversible paralysis and loss of sensory function, is among the most devastating injuries suffered by humans. A significant effort has been made over the past few decades to find potential therapies to treat spinal cord injury (SCI), an objective that faces the challenge of central nervous system regeneration. Experimental access to human spinal cord tissue is therefore a critical need for discovery of SCI therapies. More specifically, methods for the development of organ mimics known as organoids are greatly needed.

Research on spinal cord organoids has been sparse and only very recently enabled by the discovery of the necessary human progenitor cells derived from pluripotent stem cells. Herein, a human organoid model is provided that can simulate SCI in vitro and test the potential of novel therapies. The methods provided herein involve injury of the organoid with a sharp scalpel, which causes neuronal death and subsequently generates the characteristic glial scar observed after injury with its biological markers. Exposure of the injured organoid to a recently discovered pre-clinical therapy for SCI, namely bioactive peptide amphiphiles containing the sequence

IKVAV (SEQ ID NO: 1), is shown herein to avoid scar formation and promotes axon regeneration in the area of the lesion. The model developed here can be used to accelerate discovery of therapeutic strategies to treat the broad spectrum of spinal cord injuries.

### Production of Large Glia-Rich Human Spinal Cord Organoids

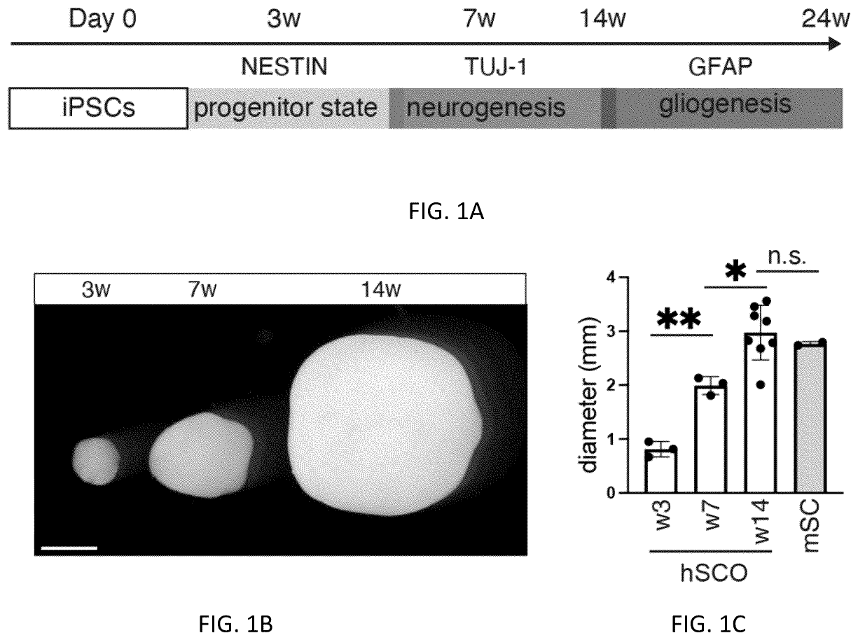

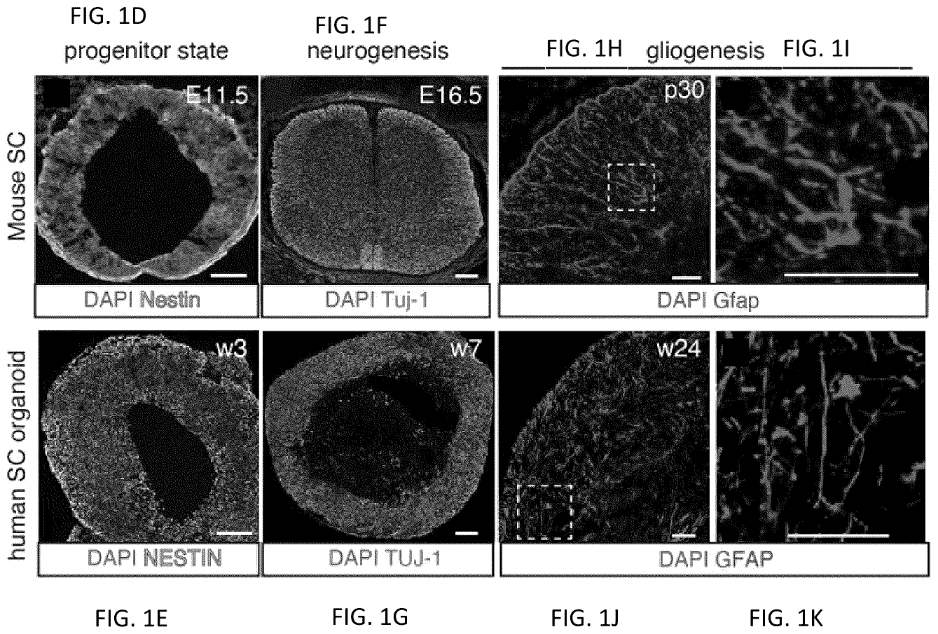

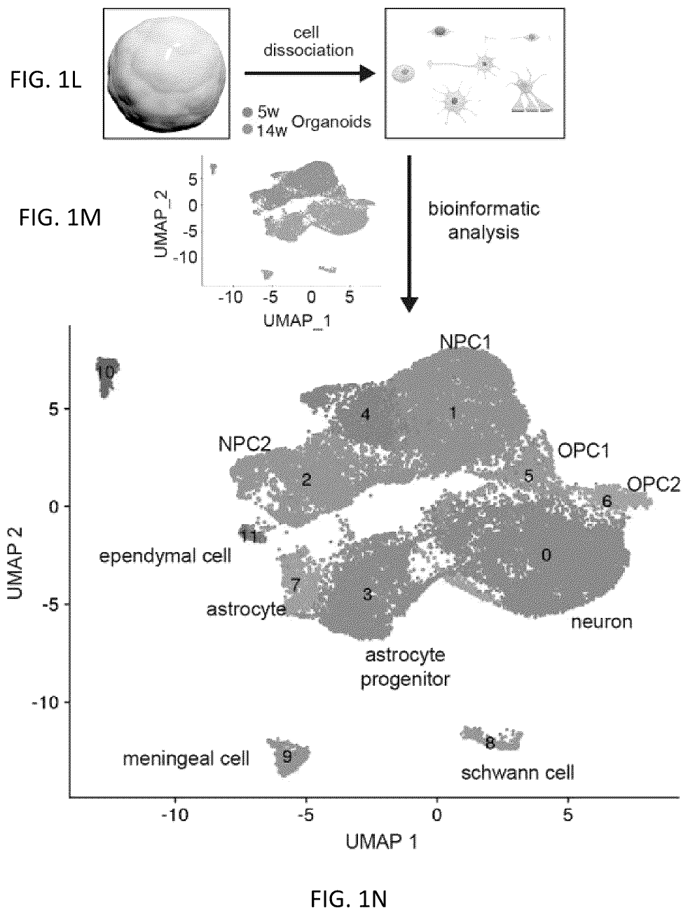

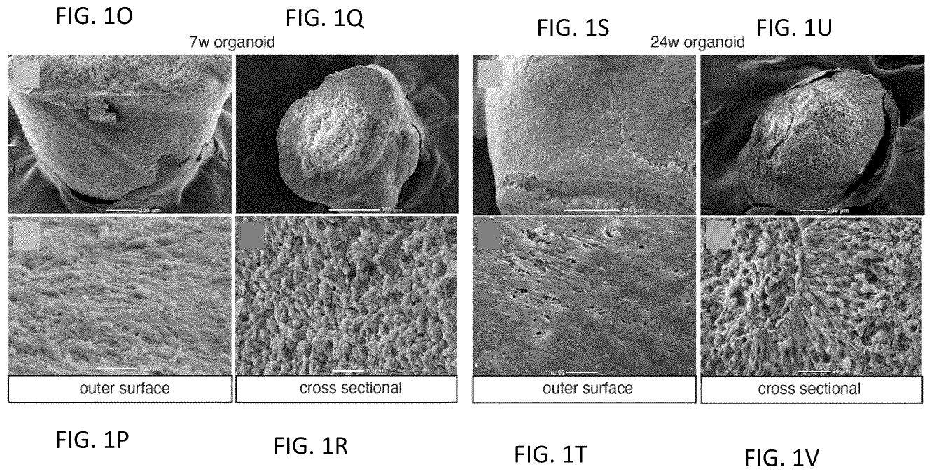

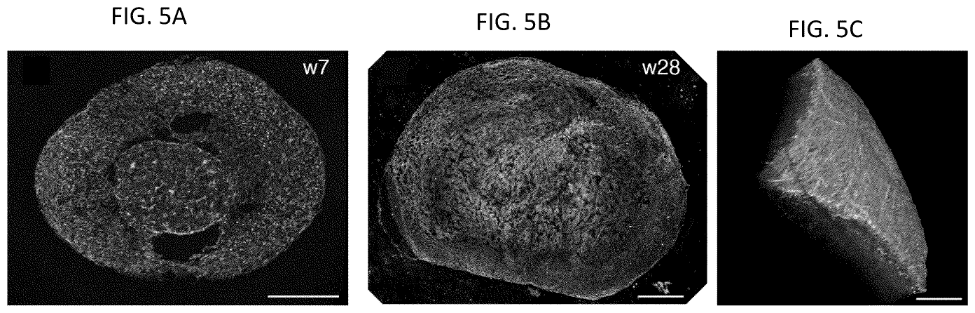

Human organoids with spinal cord cell types were generated from human induced pluripotent stem cells (iPSCs) (FIG. 1A). To monitor the tissue growth, the diameter of the organoids over time was evaluated. The 3-week-old organoid doubled in size at the 7-week stage (FIG. 1 B, C). The organoid growth continued until week 14 and achieved dimensions of about 3 mm in diameter, larger than any human spinal cord organoids reported to date, and comparable in size to the cross-section of a mouse spinal cord at postnatal day 30 (FIG. 1 B, C). Analysis of developmental biomarkers was performed to establish the transformation of NESTIN⁺ neural progenitors into TUJ-1⁺ neurons. NESTIN expression was observed after 3 weeks in culture at a level that is similar to that in an embryonic mouse spinal cord tissue at E10.5 (FIG. 1D-G) while TUJ-1 in 7-week human organoids became similar to mouse ones at E14.5 (FIG. 5A). Using an orbital shaking system over 24 weeks, the organoids matured further exhibiting outer layers containing GFAP⁺ glial cell types as observed in mouse spinal cords (FIG. 1H, J and FIG. 5B, 5C). Additionally, the morphology of these cells recapitulated those in postnatal day 30 mouse spinal cords. Furthermore, these GFAP⁺ glial cells in these human organoids acquired the lattice-like morphology characteristic of naïve astrocytes typically observed in healthy mice (FIG. 1I, K).

Single-cell RNA sequencing (scRNA-seq) was conducted. Viable single-cell spinal cord cells were isolated from 5-week and 14-week human organoids by gentle cell dissociation followed by scRNA-seq (FIG. 1L). The viable cell isolation succeeded with more than 90% survival rate evaluated by live and dead staining prior to 10X genomics droplet processing and gene library preparation. Two transcriptionally distinct populations from the two organoid stages were identified (FIG. 1M). Using next-generation sequencing and available reference data, cell subtypes present in the organoids were characterized. A comparison between data on human fetal tissues stages (Andersen, J. et al. Nat Neurosci 26, 902-914 (2023); Zhang, Q. et al. EMBO Rep 22, c52728, doi: 10.15252/embr.202152728 (2021) and the organoids demonstrated the presence of neurons, astrocytes, oligodendrocytes (myelin-producing cells), ependymal cells, meninges, Schwann cells, and neuronal as well as glial progenitor cell types (Fig. IN). However, the single-cell RNA analysis did not reveal the presence of microglia or vascular cell types.

Next, the detailed microstructure of the organoids was characterized by using scanning electron microscopy (SEM) at the two distinct stages of development. At the neurogenesis stage, the outer surface of the organoid was topographically rough, likely due to the presence of many neurons and the absence of astrocytes (FIG. 1O, P). Imaging of the 7-week organoid cross-sections revealed densely packed unorganized cells within the interior of the organoid, which are presumably less mature TUJ-1⁺ neurons (FIG. 1Q, R). In striking contrast, the 24-week organoid displayed a remarkably smooth surface covered by GFAP⁺ glial cells which are expected to have a flat morphology (FIG. 1S, T). Within the cross-section, highly organized neuronal cells that appeared to contain neurites were observed (FIG. 1U, V). These results comport with the immunohistochemical analysis shown in FIG. 1G and 1J.

### Injury of the Organoid Forms Glial Scar-Like Tissue

SCI in investigated mammals produces extensive tissue damage which in turn triggers a complex cellular and molecular cascade resulting in neuronal cell death. The lesion site structure is eventually characterized by the formation of the so-called glial scar surrounding a cavity and containing densely packed reactive astrocytes and extracellular matrix (ECM) components, a process called reactive astrogliosis. Given the similarity of cell types present in the human spinal cord organoids relative to native tissues, it was next tested whether the organoids could recapitulate some aspects of this CNS injury.

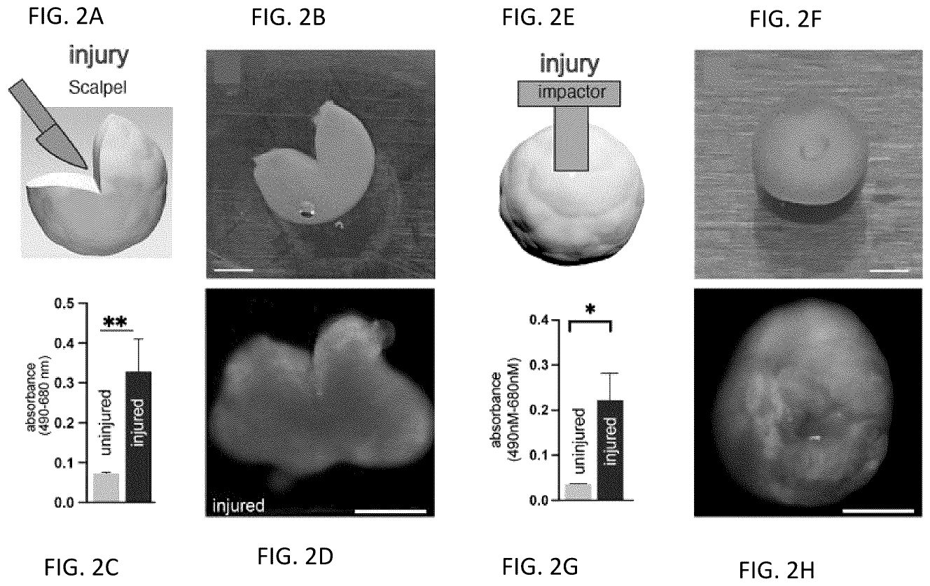

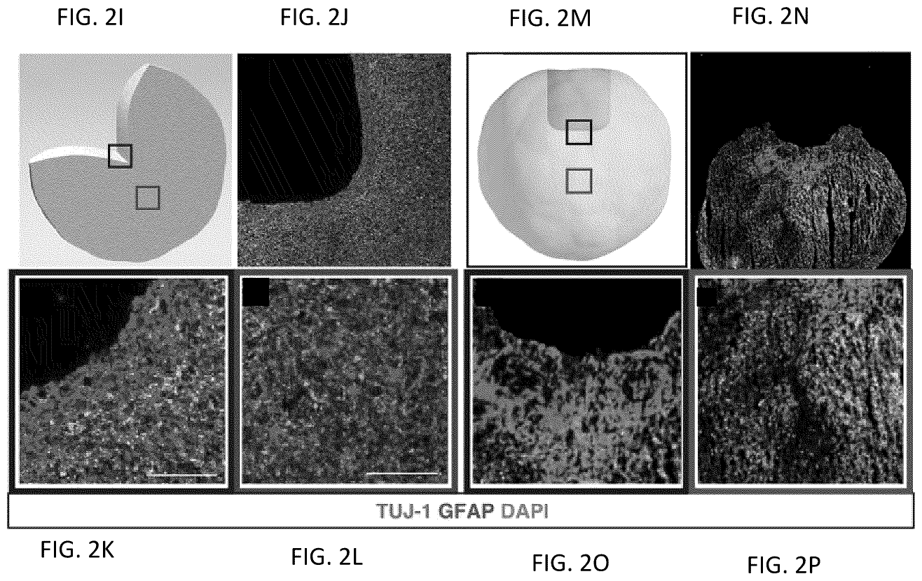

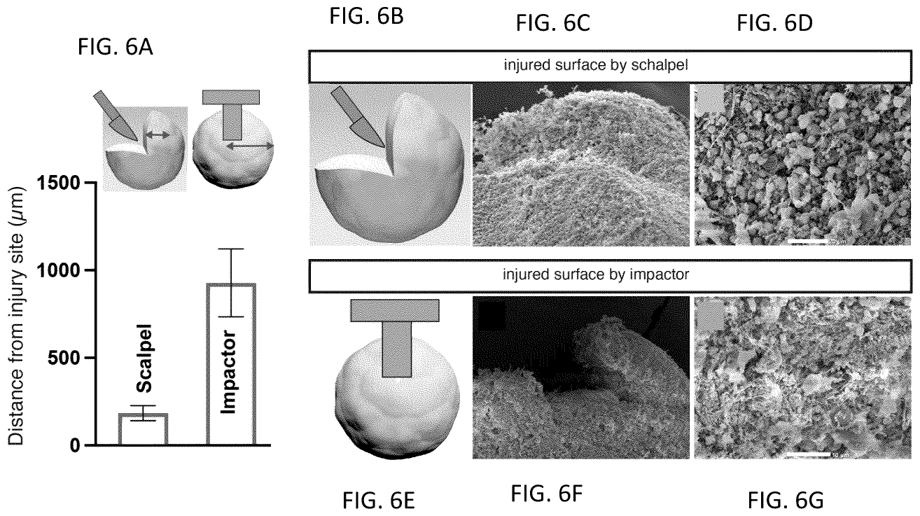

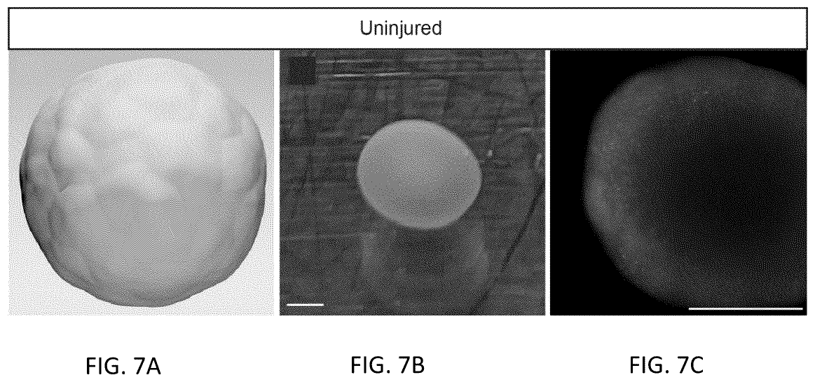

A model was developed in which a mechanical injury was produced in the organoid using a surgical scalpel, which could mimic the spinal cord hemisection often used in SCI in vivo studies (FIG. 2A). Given the clear lesion site associated with this model, injury response could be easily monitored over time (FIG. 2B). Local lesion damage was evaluated using Calcein-AM and ethidium homodimer-1 and it was found that cell death occurred specifically at the site of injury after 1 hour (FIG. 2D). This test was also used in uninjured regions of the organoid where there was no visible cell death (FIG. 7A-C). These results were further confirmed using the lactate dehydrogenase (LDH) cytotoxicity assay for injured and uninjured organoids (FIG. 2C), which demonstrates necrotic death was caused by the mechanical injury. Over time the organoid developed a glial scar-like tissue where glial fibrillary acidic protein (GFAP) was strongly expressed, and is therefore reminiscent of the typical SCI astrogliosis observed in vivo (FIG. 2I, J). Interestingly, non-injured regions showed a normal lattice-like pattern of glial cells (cells are separated from each other), whereas the GFAP+cells in the lesion were densely packed (FIG. 2K, L). These astrocytes likely migrated and proliferated to populate the injury site. SEM analysis allowed for visualization of other details of the tissue morphology revealing organized cells on uninjured surfaces of the organoid (FIG. 1V) and less organized ones on the injured organoid sites (FIG. 6A-C). It is difficult with SEM to distinguish between neurons and astrocytes, but overall significant changes in cell organization were found to have occurred as a result of the injury. The organoid injury model reproduces the initial cell death and reactive astrocyte response associated with scar-like tissue. Importantly, no obvious evidence of neuronal regeneration were seen within one month after the injury.

A second translational model for SCI which closely mimics the compressive trauma most commonly experienced by human patients was next developed. This experimental procedure in the organoid is referred to herein as the contusion model depicted by the schematic in FIG. 2E and the photograph after injury in FIG. 2F. The evaluation of the organoid injury again involved the use of Calcein-AM and ethidium homodimer-1, which clearly revealed in this case much more delocalized cell death at the site of injury after 1 hour (FIG. 2H). The LDH cytotoxicity assay (FIG. 2G) again demonstrates the occurrence of necrotic death as a result of the contusion.

Interestingly, GFAP was strongly expressed throughout the organoid even at sites distant from the external impact (FIG. 2M-P). Additionally, SEM analysis revealed less organized injured surfaces relative to healthy uninjured surfaces (FIG. 1V, FIG. 6D-6F). This contusion model is translationally useful for the evaluation of future treatments for SCI.

### Bioactive Nanofiber Treatment in Uninjured Spinal Cord Organoid

Our laboratory has developed a broad platform of bioactive biomaterials based on molecules known as peptide amphiphiles (PAs), which spontaneously create networks of nanoscale supramolecular fibers in aqueous media of high ionic strength. PA molecules in these supramolecular systems contain alkylated peptide segments with multiple domains to drive non-covalent polymerization into nanofibers that display on their surfaces signals to either activate receptors directly or bind specific signaling proteins. These PAs have demonstrated significant efficacy in regeneration of different tissues such as spinal cord, muscle, bone, and cartilage, as well as blood vessels in pre-clinical models, promote the selective and rapid differentiation of neural stem cells into neurons as well as human motor neuron maturation in vitro. These matrices also led to significant regeneration in the injured spinal cord in vivo based on both histological evidence and functional recovery (Tysseling-Mattiace, V. M. et al. J Neurosci 28, 3814-3823, (2008); Alvarez, Z. et al. Science 374, 848-856, (2021).

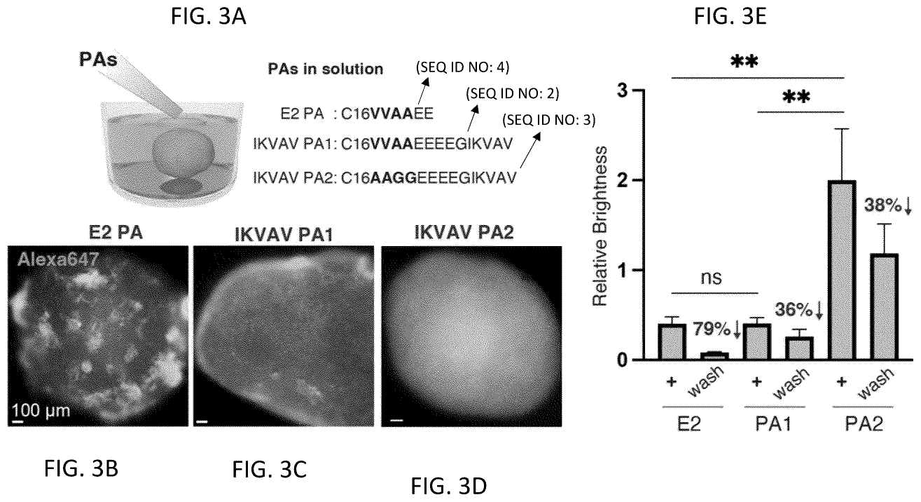

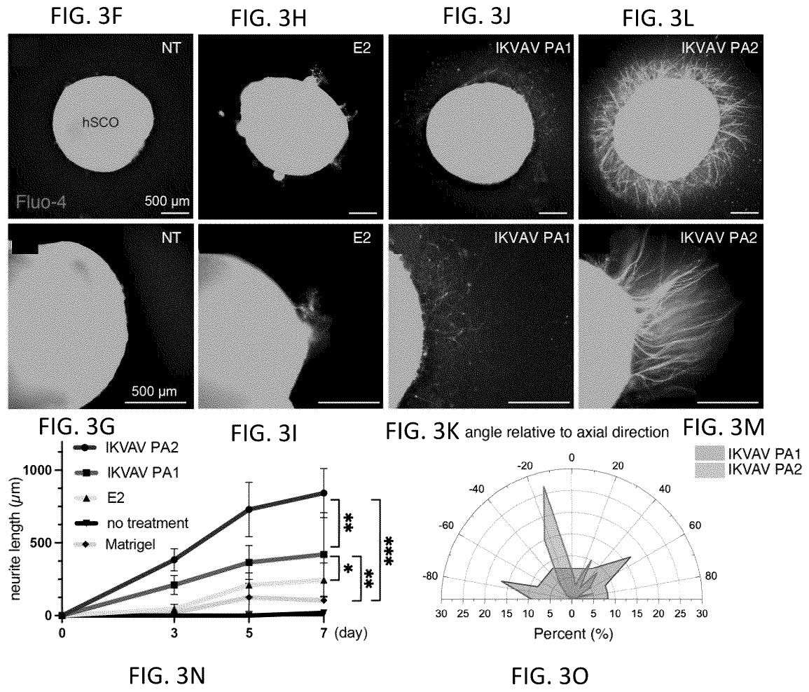

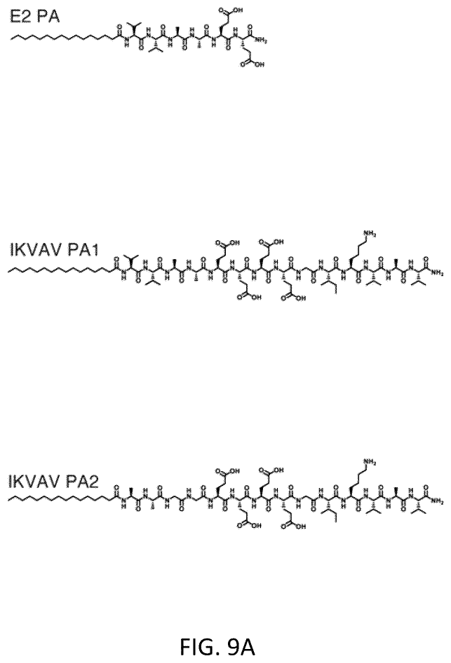

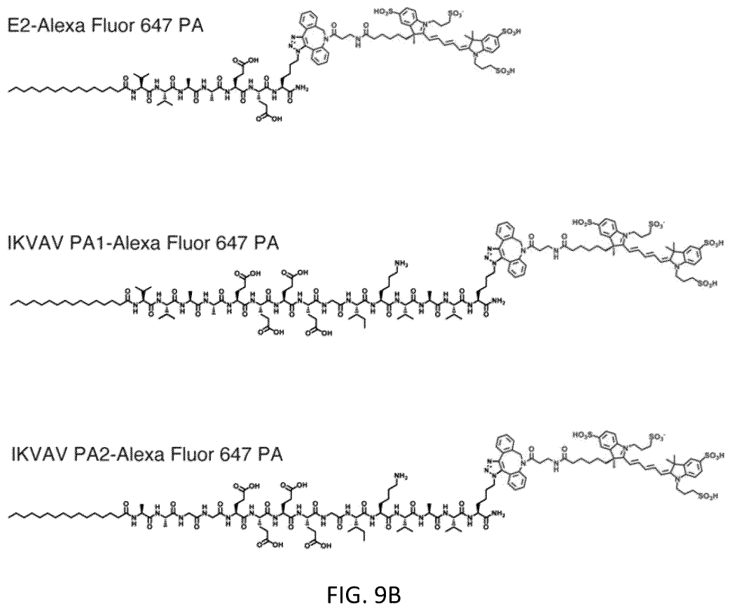

Two different nanofibers comprising the bioactive peptide IKVAV (SEQ ID NO: 1) have been developed that exhibit either slow (PA1) or fast (PA2) internal supramolecular motion. PA1 and PA2 are understood to include the bioactive peptide IKVAV (SEQ ID NO: 1), and are also referred to herein as “IKVAV PA1” and “IKVAV PA2”. The most dynamic system (PA2) facilitates differentiation of neural stem cells into neurons and enhances axonal regeneration after injury in vivo. These two systems were tested on our human spinal cord organoids herein (FIG. 9A-9B). A solution of the PA nanofibers comprising IKVAV (SEQ ID NO: 1) as well as a PA in saline to the media of uninjured 7-week organoids, which did not contain glial cells (FIG. 3A), was tested. Using PAs fluorescently labeled with Alexa647, interactions of the nanofibers with the organoid surfaces was investigated. Organoids treated with a PA control which does not contain the IKVAV (SEQ ID NO: 1) signal (E2 PA) formed patchy deposits of nanofibers that wash off easily after exchanging media. This indicates a very weak interaction between the PA and the organoid surface. Treatment with PA1 also revealed patchy adsorption which does not wash off as easily as E2 PA (FIG. 3B, C, E). In great contrast, PA2 homogeneously covered the organoid surface with strong fluorescence signals (FIG. 3D, E). Based on the fluorescence signals the percentage of each PA that is removed after the washing step was calculated. The percentage removed after the washing step was 79%, 36% and 38% in E2, PA1 and PA2, respectively (FIG. 3E). This results demonstrate a relatively fast adhesion of the most bioactive PA (PA2) to the spinal cord organoid.

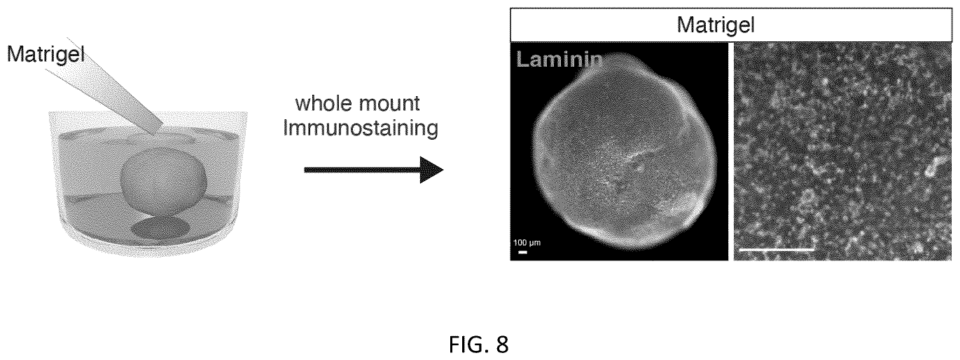

Next outward neurite growth from the organoid surfaces into the media was investigated (FIG. 3F-M). After 7 days, small protrusions of neurite aggregates emanating from the surface of organoids exposed to E2 PA nanostructures which lack the bioactive signal were observed. In the presence of PA1 in the media, which creates nanostructures with slow supramolecular motion of the bioactive molecules, distinct filamentous extensions of neurites after the same time period were observed. Strikingly, PA2 nanofibers in the media, which formed the most bioactive scaffold in vivo using a pre-clinical spinal cord injury model, revealed much longer and denser arrays of the neurite extensions emanating perpendicular to the surface of the organoid. This observation in the human spinal cord organoid validates the functional and histological efficacy of the therapy that reversed paralysis in the mouse model. in vivo it had been observed that PA2 scaffolds led to axonal extensions across the injury site, a decreased level of glial scarring, growth of new blood vessels in the spinal cord, as well as enhanced survival of motor neurons and myelination. Since Matrigel is the common commercially available biological matrix used in the growth of organoids, it was added soluble form to the media and observed very limited protrusions of neurites, in fact more comparable to those observed upon addition of E2 PA (FIG. 8).

The rate of neurite extension from the surface of organoids was measured, which was found to increase with elapsed time after the addition of treatment to the media (FIG. 3N). However, the measured neurite growth speed was found to be 4.46 μm, 1.79 μm and 1.07 μm per hour in PA2, PA1 and E2 PA conditions, respectively. This observation correlates well with the relative bioactivity of these three systems. Furthermore, a very interesting finding when PA2 treatment was used was an enhanced alignment of neurites as their growth occurred perpendicular to the organoid surface. In order to quantify this phenomenon, a polar histogram was generated. Interestingly, the rapidly growing extensions using the more dynamic bioactive PA led to a higher degree of neurite co-alignment (FIG. 3O). The rapid growth of neurites may lead to formation of denser arrays, and therefore naturally to self-organization of the axons perpendicular to the surface. This is analogous in fact to the self-ordering of high aspect ratio molecules in liquid crystalline phases in nature.

### PA Nanofiber Treatment of Injured Spinal Cord Organoid

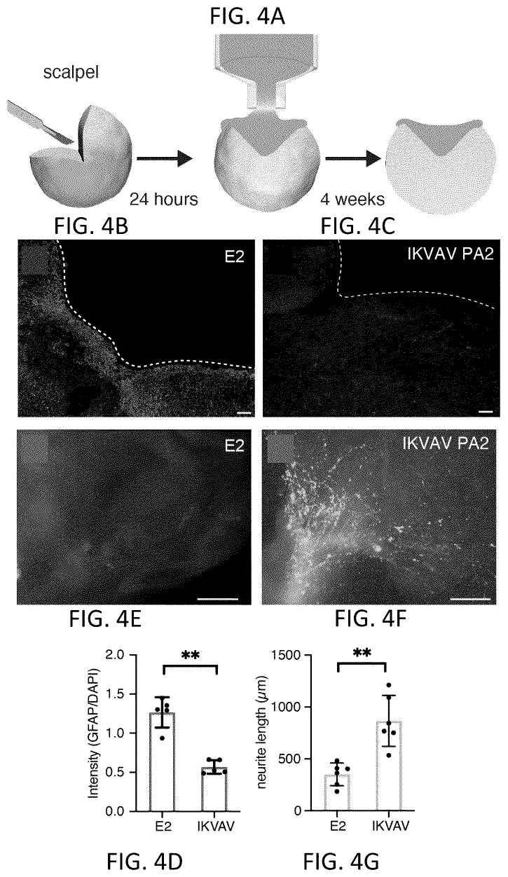

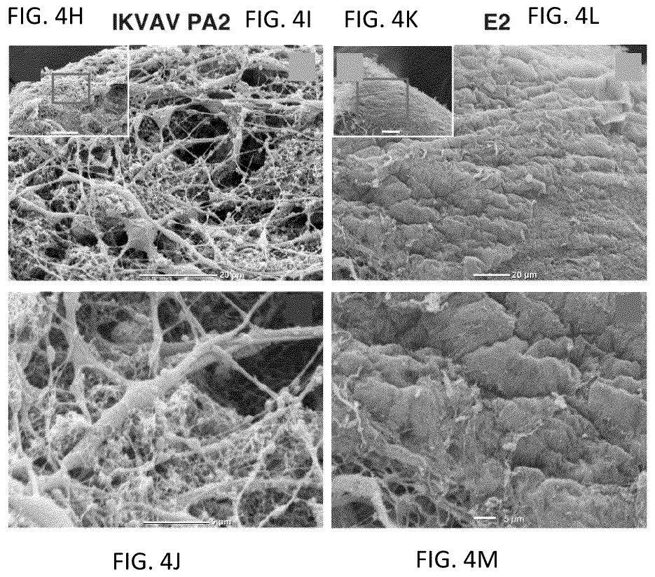

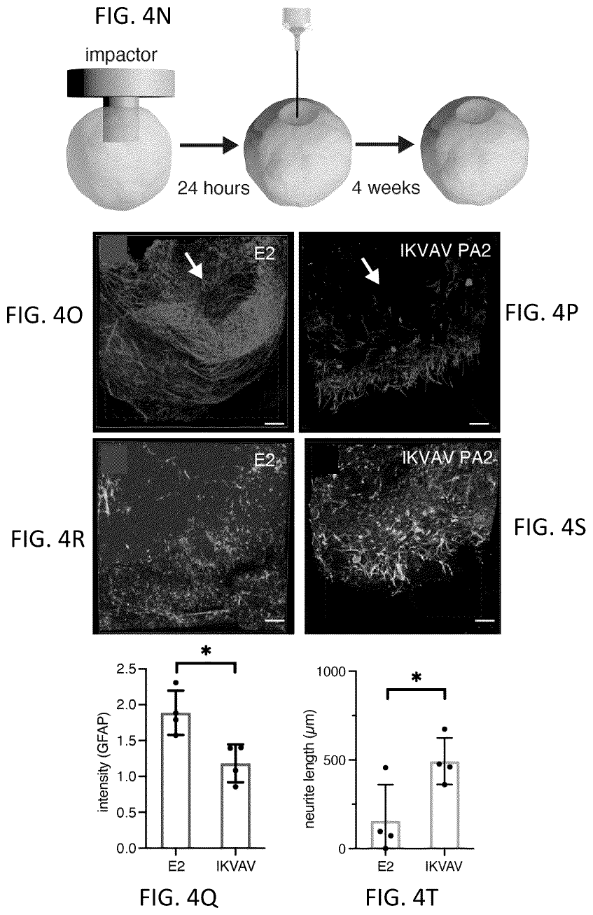

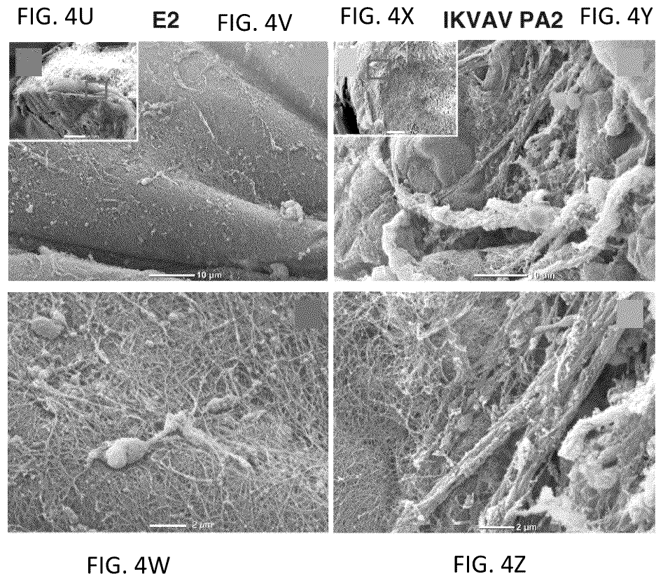

Next PA2 was used as a therapy mimic on the injured organoid (FIG. 4A). Based on previous in vivo experiments in murine models, it was hypothesized that these scaffolds with intense supramolecular motion could promote axonal growth from neurons within the injured organoid. In recent SCI animal models, nanofibers formed by IKVAV PA2 that are dissolved in saline undergo gelation into a localized scaffold when injected at the site of injury. This phenomenon is understood as the result of significant electrostatic screening of the negatively charged nanofibers by ionic species present in physiological fluids. The injured surface of the organoid was thus treated with an IKVAV PA2 solution dispensed from a pipette. The instantaneous formation of a hydrogel covering the lesion cavity was observed (FIG. 4A). A similar experiment was performed using nanofibers formed by E2 PA which lacks the IKVAV bioactive epitope. One month after treatment of the scalpel-induced injury, organoids treated with E2 PA nanostructures revealed the same glial response observed in untreated but injured organoids (FIG. 4B). In great contrast, those exposed to IKVAV PA2 exhibited a dramatically reduced formation of this GFAP+scar-like tissue near the injury site (FIG. 4C, D). Furthermore, Fluo-4 live staining of this treated group revealed the growth of long neurites emanating from spared neurons after injury into the scaffold (labeled with Alexa Fluor 647 dye) (see FIG. 4F and G). The long neurites observed in FIG. 4F also reveal a punctate appearance which we believe could be due to the presence of cellular debris. This debris may be present as a result of dead cells produced by the injury to the organoid given that immune cells are not available for phagocytic function in this model. The presence of long neurites is consistent with axonal extension previously observed in vivo upon treatment of the injury with the bioactive PA, which significantly activates β1-integrin receptor. In contrast E2 PA nanofibers, which can provide some mechanical structural support similar to natural ECM, showed only short neurites growing into the gel space (FIG. 4E, G). This difference is clearly due to the high level of bioactivity offered by IKVAV PA2 supramolecular nanofibers. To visualize the obvious difference in response to organoid therapeutic treatment, SEM imaging of the injured organoid surfaces exposed to the two different conditions was performed. Interestingly, SEM revealed the presence of neurite-rich cellular structures in the organoids treated with the bioactive PA (FIG. 4H-J). The diameter of PA nanofibers is typically about 10 nm, and therefore they could be distinguished from the much larger micron-scale diameters of axons extended by neurons (FIG. 4J). An additional feature observed by SEM is the close interaction between PA nanofibers and the newly formed axons on the injured surfaces of organoids. Small particles are observed on and around the neurites in the SEM images. This supports the earlier suggestion that uncleared cellular debris is present after cell death resulting from the injury. In great contrast to organoids treated with bioactive IKVAV PA2, FIG. 4K, L shows similar micrographs of injured organoid surfaces that were treated with non-bioactive E2 PA. In this case the significant number of neurons and neurites visualized in FIG. 4K-M, was not observed, and instead a smoother texture was observed that could be consistent with the presence of glial cells on the injured surface revealed by fluorescence microscopy.

The efficacy of the bioactive nanofiber therapy using the organoid contusion model of SCI described above (FIG. 4N) was investigated. Whole-mount immunostaining on the contusion surface revealed localized GFAP⁺ astrocytes one month after treatment with the E2 PA control (FIG. 4O). In contrast, the GFAP⁺ biomarker was reduced following treatment with the bioactive IKVAV PA2 (FIG. 4P, Q). Furthermore, organoids treated with E2 PA showed only a few Fluo-4⁺ cells (FIG. 4R), whereas Fluo-4⁺ cells appeared to be growing into the bioactive system IKVAV PA2 (labeled with Alexa Fluor 647) (FIG. 4S, T). SEM imaging of the injury site in the

E2 PA treated samples revealed the presence of only a few neurons with short axons in contact with the gel (FIG. 4U-W). In contrast, neurons penetrating the nanofibers gels were clearly visible in the case of IKVAV PA2 treated contusions (FIG. 4X-Z). Given the agreement of these observations with earlier in vivo work on SCI (Tysseling-Mattiace, V. M. et al. J Neurosci 28, 3814-3823 (2008); Alvarez, Z. et al. Science 374, 848-856), these human spinal cord organoids are extremely useful for the discovery and validation of novel therapies for SCI.

### Discussion

SCI is clearly one of the most devasting survivable human injuries and its effective treatment at the acute or chronic levels has remained elusive. An intense search is ongoing around the world seeking potential therapies with limited success to date and it is therefore critical to create more translational discovery strategies to benefit patients. In this context, described herein are two injury models and therapeutic treatment of a “human” spinal cord organoid that are consistent with results with a recent in vivo murine model of SCI (Alvarez, Z. et al. Science 374, 848-856, doi: 10.1126/science.abh3602 (2021). This recent work uncovered a novel molecular phenomenon that resulted in highly positive outcomes from the pre-clinical murine model. The approach here to the human injury using the organoid was to produce laceration using a sharp scalpel or a compressive contusion commonly used in SCI research, which actually mimics the human injury. The therapies investigated are filamentous supramolecular nanostructures formed by self-assembly of bioactive molecules that target the β1-integrin receptor. The initial experiments exposed an uninjured organoid to these nanoscale filaments in growth media that clearly revealed a greatly enhanced level of axonal growth on the surface of the organoid when the molecules used promoted intense supramolecular motion. Furthermore, the extensive axonal growth normal to the organoid surface clearly led to self-alignment of the neurites. Conversely, suppression of the supramolecular motion in very similar bioactive filaments resulted in a minor amount of axonal growth consistent with previous in vivo results. The injured organoids treated by the most bioactive nanostructures revealed extensive axonal regeneration into the gel network placed on injured surfaces and used for treatment in in vivo work. Furthermore, a remarkable reduction in the formation of glial scar-like tissues was observed, which is again consistent with previous in vivo results and a very important target for novel therapies that could raise patient functionality. This approach provides a simple platform that will be useful in the discovery of treatments for both the acute and chronic injuries.

### Example 3

Maintenance and evaluation of human induced pluripotent stem cells

The following exemplary methods related to human inducted pluripotent stem cells can be used, including in the development of the organoids described herein. iPSCs are cultured in mTeSR or StemFlex Medium on Matrigel coated plates in CO₂ incubators. The cells are fed fresh mTeSR every day. When thawed or split, cells are cultured in mTeSR supplemented with ROCK inhibitor (RI) to prevent death dance-mediated apoptosis. The cells are split with EDTA for passaging. For dish coating, 100 μL of growth factor reduced Matrigel is added into 4 mL of pre-chilled DMEM/F12 and kept on ice. The mixture is added into 6 well-tissue culture dishes and incubated for 20 mins at Room temperature under a sterile condition in a biosafety cabinet. When the incubation is done, the DMEM/F12/Matrigel is discarded and washed once with PBS before the stem cell maintenance medium is added.

The stem cell maintenance medium supplemented with 10 μM RI and DMEM/F12 media is warmed. For EDTA dissociation, media is aspirated from plates and washed once with PBS. 1 mM EDTA/PBS is added to the plate. The plate is placed in an incubator at 37° C. for 5 mins. The EDTA is gently aspirated while cells remain attached to the dish. Gently pipette up-and-down with warm DMEM/F12 to remove the cells from the plate and break up large chunks. These cells can then be directly passages to the new plate containing mTeSR+RI at the desired density (e.g., at 1 in 10 dilutions). The rest of the cells can be frozen for long term storage. Prepare freezing medium as follows: 10% DMSO/40% FBS/50% mTeSR. Resuspend 10⁶ cells per tube in the freezing medium. Put tubes first at −80° C. for 24 hours and then liquid nitrogen for long-term storage. Validation of hiPSCs is performed every 3 months or 10 passages. For example, stem cell activity can be visualized under a light microscope, and/or fluorescent immunostaining with pluripotent stem cell markers can be performed. The characteristics of cell morphology and colony shape can be also evaluated. F-ACTIN staining enables morphological evaluation. The cells are discarded if there is any evidence of a significant reduction in those gold standard markers. The morphological abnormality is typically associated with a non-homogenous cell population partially due to contamination of spontaneously differentiated cells.

### Example 4

Directed Differentiation of iPSCs by Defined Factors

Experiments were conducted during development of embodiments herein using iPSCs 18a, one of the available cell lines known to generate functional neurons with a range of efficiencies similar to ESCs as a robust cell resource to generate spinal cord neurons. Exemplary methods for guiding differentiation of iPSCs, including in the generation of the organoids described herein, are described below.

Aspirate stem cell media from plates and wash once with PBS. To dissociate undifferentiated iPSCs to single cells, add to the culture an enzyme, 1 mM EDTA/PBS for 5 mins at 37° C. Gently pipette up-and-down with warm DMEM/F12 supplemented with 10 μM ROCK inhibitor (RI) to collect the cells into a conical tube followed by centrifugation.

Count the cell number manually in a blood count chamber to exclude dead and blebbed cells stained by trypan bluc. Seed in low-cell-adhesion U-bottomed 96 wells (Sumitomo Bakelite Co., Ltd. #MS-9096UZ) to reaggregate cells quickly at a density of 10,000 cells/well, 100 μl in a following medium condition: Spinal cord induction medium (SCIM) containing N2B27 [DMEM/F-12, neurobasal medium (1:1), 0.5% N2 supplement and 1% B27 supplement without vitamin A, 10% knockout serum replacement] supplemented with 1 mM Glutamax, 0.1 mM 2-mercaptoethanol, 0.5 μM ascorbic acid and 100 U/ml penicillin, and 100 mg/ml streptomycin. Defining the day on which the differentiation culture was started as day 0.

From day 0 to day 3, 10 μM RI, 20 ng/ml recombinant human basic FGF, 3 μM CHIR99021 and 10 μM SB431542 were added into SCIM and use 100 μL per well (Sumitomo Bakelite Co., Ltd. #MS-9096UZ). From day 3 to day 6, add 100 μL SCIM supplemented with 10 μM SB431542 and 100 nM retinoic acid (RA) per well (Sumitomo Bakelite Co., Ltd. #MS-9096UZ). (optional for full medium change) From day 3 to day 6, the growing organoids are transferred into 10 cm petri dish and washed with N2B27 to eliminate the initial instructional growth factors. Next, transfer the organoids into the low-cell-adhesion U-bottomed 96 wells containing SCIM supplemented with 10 μM SB431542, 0.5 M SAG (Enzo Life Sciences, Inc, #ALX-270-426-M001) and 100 nM retinoic acid (RA). From day 6 to day 15, perform half medium change with SCIM supplemented with 0.5 μM SAG (Enzo Life Sciences, Inc, #ALX-270-426-M001) and 100 nM retinoic acid.

Validation of spinal cord progenitor organoids (SPOs) is performed. The SPOs are fixed in 4% PFA for o/n at 4′C. The tissues are then cryoprotected in 30% sucrose/PBS for o/n at 4° C. A cryostat is used to obtain 10 μm tissue section on Superfrost™ Plus Microscope Slides (Fisherbrand™ 22-037-246). Fluorescent immunostaining was performed on the cryo-sectioned slides to visualize NESTIN. Spinal cord tissue identity can be evaluated by total RNA harvested from organoid tissues via purification by Qiagen RNeasy Micro Kits. High resolution transcriptomic analysis can be performed to identify each cell subtype in greater detail.

### Example 5

### Long-Term Culture of Spinal Cord Organoids Until 180 Days

Spinal cord progenitor organoids (SPOs) can be further cultured and matured into human spinal cord organoids. Exemplary methods for maturing the SPOs into human spinal cord organoids are described below.

Once the spinal cord identity is acquired within organoids, brain trophic factors used were added to a maturation medium. Culture agitation on an orbit shaker in a tissue culture incubator allows for homogenous nutrient/gas support and protect SPOs from local waste-product accumulation. On day 15, the SPOs were transferred to a 10 cm Petri dish and washed with N2B27 medium to eliminate the instruction factors in the media. Then, the tissues are cultured in spinal cord maturation medium (SCMM) containing N2B27 medium supplemented with 1 mM L-Glu, 0.1 mM 2-ME, 0.5 μM ascorbic acid, 10 ng/ml BDNF, 20 ng/ml GDNF and 100 nM RA. 6 wells dishes coated non-adhesion solution are used. Typically, 5 organoids per well with a small volume (about 1.5 mL) medium result in better outcomes as far as avoiding tissue-tissue fusion. Perform half-medium change with SCMM. The orbit shaker is used to agitate the culture. Validation of spinal cord organoids (SCOs) is performed. The SCOs are fixed in 4% PFA for o/n at 4° C. The tissues are then cryoprotected in 30% sucrose/PBS for o/n at 4° C. A cryostat is used to obtain 10 μm tissue section on Superfrost™ Plus Microscope Slides (Fisherbrand™ 22-037-246). Fluorescent immunostaining can be performed on cryo-sectioned slides to visualize TUJ-1 and GFAP. Spinal cord identifies can be evaluated by total RNA harvested from organoid tissues via purification by Qiagen RNeasy Micro Kits.

The initial phase of neuronal differentiation produces neuron-specific tubulin, TUJ-1 positive cells characterized as morphologically distinct from the progenitors. Those TUJ-1 populations acquire cell-type-specific characteristics throughout the CNS neurogenesis. Whereas SOX2-positive neural progenitors began to segregate from TUJ1-expressing neurons at mouse E12.5, immunohistochemical analysis herein shows that one-month-old human spinal cord organoids expressed TUJ1-rich regions in the outside of the progenitors. During neurogenesis, the number of organoids per well and the volume of the medium to avoid tissue-tissue fusion is limited. 5 organoids can be cultured in 1.5 mL and 3 mL in 6 wells until 30 days and 100 days, respectively. Alternatively, transfer organoids into 24 wells individually and keep culture 0.5 mL with half medium change every three to five days.

Experiments were conducted during development of embodiments herein to direct the human spinal cord organoids into glia. Oligodendrocytes and astrocytes in mice appear at late developmental stages. The mouse spinal cords at E16.5 and postnatal day 30 were dissected.

Three months old human spinal cords showed a few GFAP⁺ cells located in the outer layer, reminiscent to postnatal day 1 mouse spinal cords. The expression level of GFAP and the number of positive cells increases over time. In summary, 6-month-old organoids showed a robust glia layer covering the neuronal region similar to the mouse-tissue counter parts.

### Example 6

### Neuroimmune Spinal Cord Organoids

Microglia are the resident immune cells and the most sensitive responders to CNS injury, actively surveilling the environment, as well as reacting to local tissue damage and systemic inflammatory cues. Neuroinflammation after injury is responsible for astrocyte reactivity, which leads to neurotoxic responses. Furthermore, microglia-dependent neuroinflammation regulates glial scarring in a contusion SCI model. It was tested herein whether neuroinflammatory components in organoids would lead to better models of SCI.

In order to introduce microglia in the spinal cord organoids, two different approaches were used. In the first approach (microglia assembly pathway 1), developmental infiltration of microglia progenitors into the SC organoid was recapitulated (FIG. 10A). To obtain the progenitors, a developmental protocol to differentiate the same iPSCs used to generate the original SC organoids into erythro-myeloid progenitors (EMPs) was performed. The EMPs were then added to the media of the spinal cord organoids (SCOs) using low-adhesion 96-well plates (Sumitomo Bakelite Co., Ltd. #MS-9096UZ) (FIG. 10B,C) to mimic microglia in the native fetal mouse spinal cord (FIG. 10D). Using EMPs labeled with CellTracker, the initial localization of the these cells on the surface of the organoids within three days was observed (FIG. 10E, F). Interestingly, such co-culturing led to the expression of the marker IBA-1 indicating the presence of mature microglia derived from the progenitors (FIG. 10G). Subsequent use of flow cytometry revealed that close to 4.4% of the cells were double positive for CD45+and CD11b+which indicate these cells are indeed microglia (FIG. 10H, I).

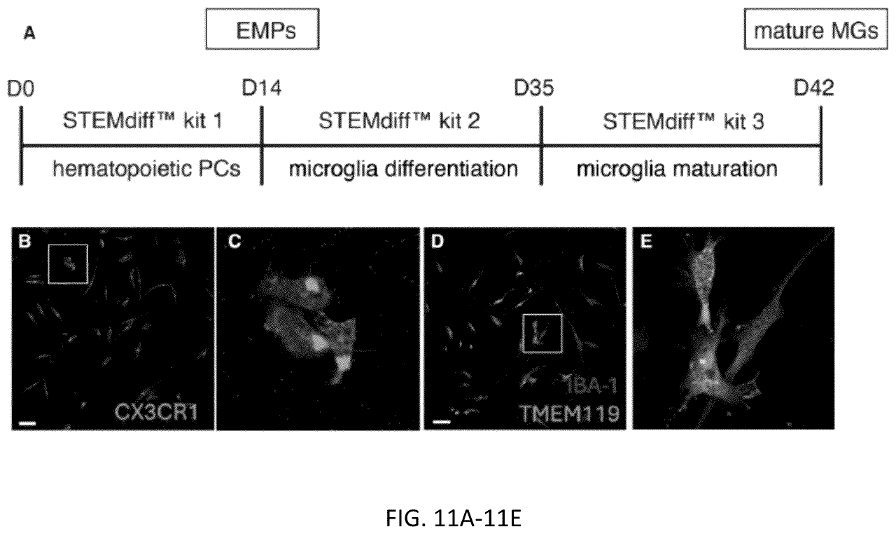

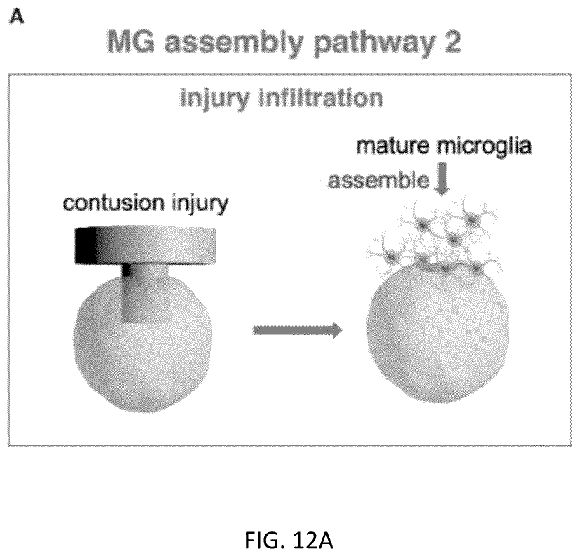

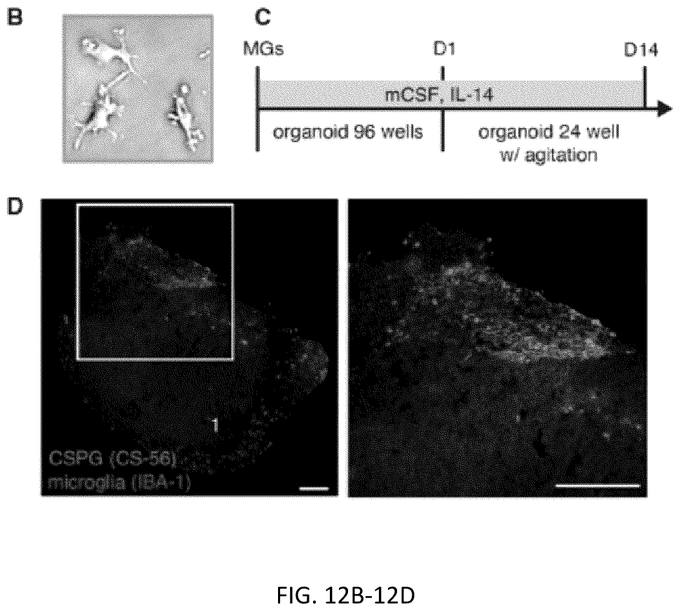

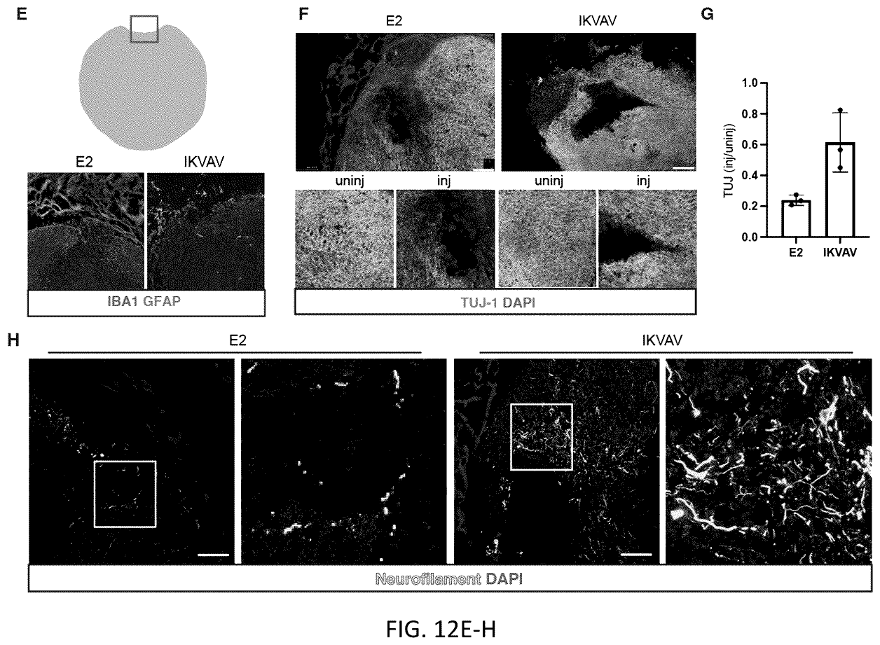

In the second approach (microglia assembly pathway 2), freshly injured SC organoids were used and infiltration of IBA1-positive mature microglia differentiated using a standard protocol (FIG. 11A-E) was allowed. In order to match the abundance of microglia reported previously in a mouse model of SCI (10-20% of cells), ˜80,000 mature microglia were added to the organoids immediately after contusion injury (FIG. 12A) and cells were allowed to infiltrate the SCOs. After exposure of the cells to the injured organoids the media was supplemented with microglial survival factors mCSF and IL-34 (FIG. 12B, C). Immunostaining for IBA-1 (microglia), GFAP (astrocytes), and chondroitin sulfate proteoglycans (CSPGs) detected by CS-56 antibody, confirmed of the presence of all three markers within the injured organoid tissue (FIG. 12D).

To observe the injury-specific response in microglia-containing organoids produced by microglia assembly pathway 1, the contusion injury was performed on the co-cultured organoids. C1QA, an injury response marker associated with the complement system and produced by activated microglia (also known as MI microglia), was detected. Interestingly, the presence of complement component 3 (C3) (FIG. 10J-K), a characteristic marker for reactive astrocytes (also known as A1 astrocyte), was also observed. These biomarkers are associated with immune responses after SCI. The injured organoids were then treated with PA or control. Significant neurite growth in the organoids treated with IKVAV PAs compared to the E2 control was observed (FIG. 10L, M). Histological analysis also revealed IKVAV PA treatment led to the significant expression of neurofilament protein which is an axon marker (FIG. 10N). To determine if PA therapy changed the markers associated with glial scar formation, organoids were stained for GFAP. Elevated expression of GFAP was observed in the injured versus uninjured areas, which is consistent with the presence of reactive astroglia (FIG. 10O-Q). The IKVAV PA treatment was also found to reduce the formation of glial scar-like tissue in the area injured by contusion. The inflammation-dependent marker CSPG was also investigated, and a greater increase around the injured tissue was observed (FIG. 10R, S). Interestingly, the CSPG expression was markedly diminished by the treatment with IKVAV PA nanofibers to nearly the same level as that of uninjured areas (FIG. 10R, S).

The injured organoids containing mature microglia introduced through microglia assembly pathway 2 were also characterized. Immunostaining was performed in these organoids for IBA-1. Cells positive for this marker in the lesion area where GFAP was also enriched were observed (FIG. 12E). Interestingly, these IBA-1+cells disappeared when organoids were treated with IKVAV PA (FIG. 12E), suggesting that the therapy diminishes the local inflammatory response. To further evaluate the potential therapeutic effect of IKVAV PA, organoids were stained for neuronal-specific markers, including TUJ-1 and neurofilament protein. TUJ-1 expression was elevated around treated lesions (FIG. 12F, G), suggesting greater survival of neurons present in injured and treated areas. Staining for neurofilament protein generated evidence for greater axonal regeneration in organoids treated with the bioactive IKVAV PA relative to the E2 control (FIG. 12H). Furthermore, to investigate the glial cell response at the transcriptome level, bulk-level RNA sequence data collected on organoids injured through pathway 1 and treated with either IKVAV

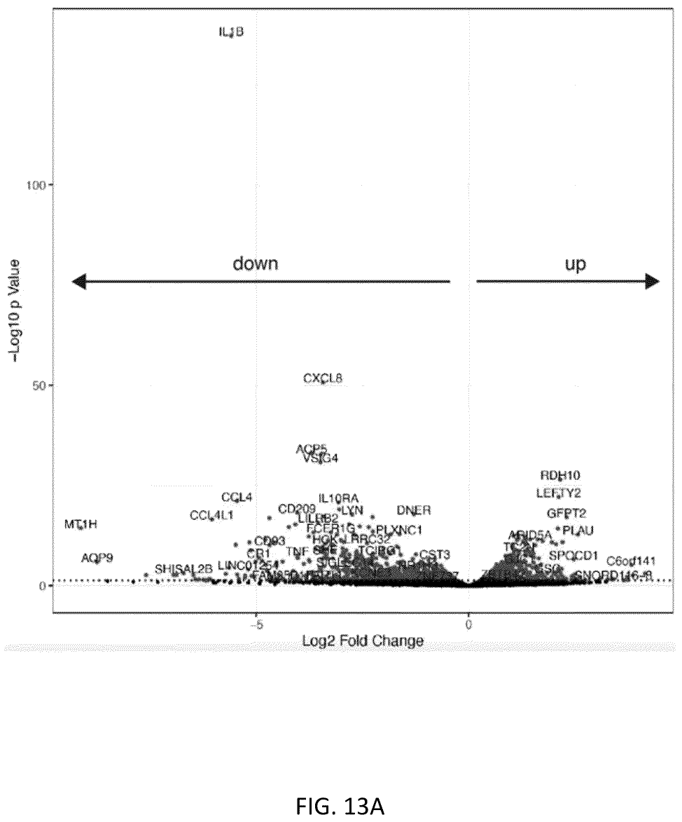

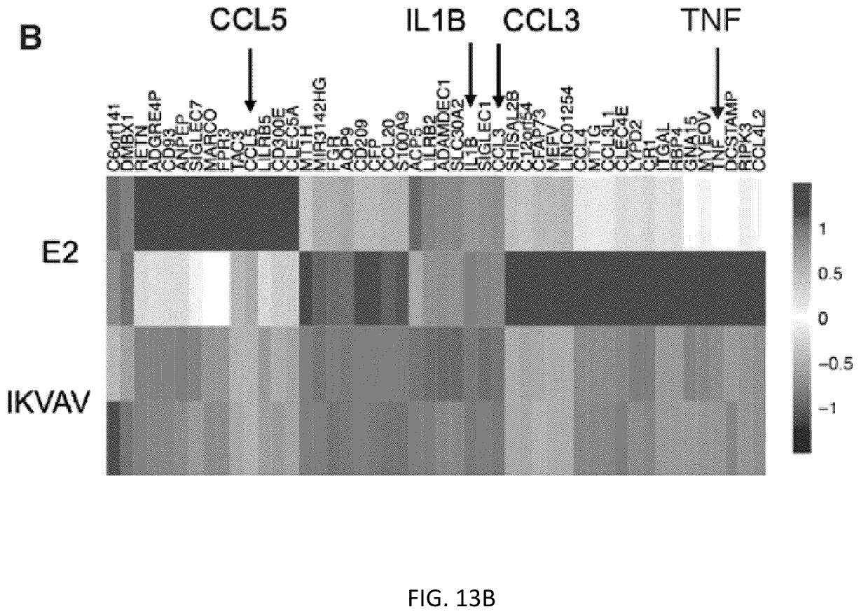

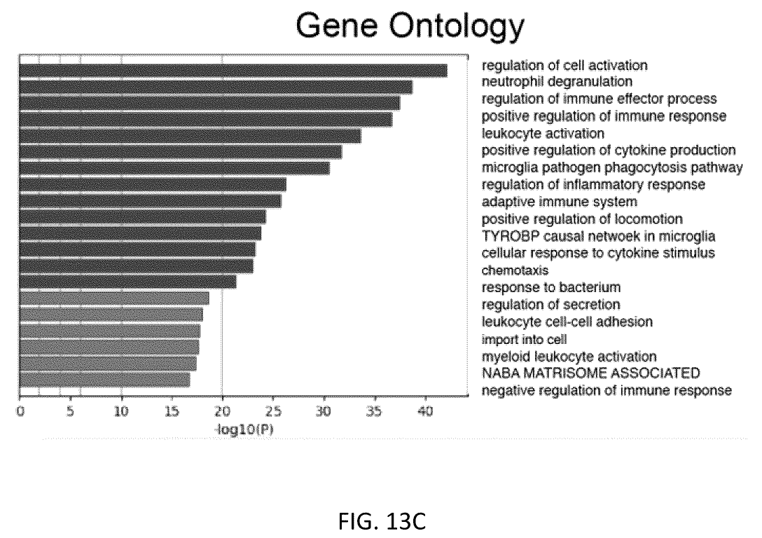

PA or the non-bioactive E2 control was obtained. The volcano plot analysis revealed different gene expression profiles in response to the PA treatment (FIG. 13A). Interestingly, the 50 top ranked genes extracted from the plots showed that several SCI related pro-inflammatory genes were downregulated in response to IKVAV PA relative to the control (FIG. 13B, C).

In both neuroimmune organoid models, CSPG and GFAP were present after injury (FIG. 10O-S), and untreated organoids showed poor neuronal regrowth (FIG. 10N). The presence of abundant CSPGs and glial scar-like tissues suggests that organoids exhibit mature characteristics expected in adult cords. This is significant as cord tissues that do not exhibit mature characteristics (e.g. neonatal cord tissues) display scar-free regeneration and spontaneous neuronal regrowth that is not observed in adult cords. Moreover, neonatal cord tissues show little deposition of CSPG and only a modest accumulation of GFAP positive cells at the lesion site. As such, the organoids herein are significantly more accurate models of spinal cord injury in adult subjects.

1. A human spinal cord organoid (hSCO) comprising neural cells, astroglial cells, and immune cells.

2. The hSCO claim 1, wherein the immune cells comprise microglia, macrophages, neutrophils, T-cells, or a combination thereof.

3. The hSCO of claim 2, wherein the immune cells comprise microglia.

4. The hSCO of claim 3, wherein the neural cells express class III beta-tubulin (TUJ-1), the microglia express IBA-1, and the astroglial cells express GFAP.

5. The hSCO of claim 1, further comprising oligodendrocytes and/or ependymal cells.

6. The hSCO of claim 1, wherein the hSCO is substantially spherical in shape and has an average diameter of at least 2 mm.

7. The hSCO of claim 1, wherein the hSCO has an average diameter of at least 2.5 mm.

8. The hSCO of claim 1, wherein the astroglial cells are present on an outer surface of the organoid, and wherein the outer surface is substantially smooth.

9. The hSCO of claim 1, wherein the hSCO has been cultured for at least about 8 weeks.

10. The hSCO of claim 9, wherein the hSCO has been cultured for about 8 weeks to about 28 weeks.

11. The hSCO of claim 1, wherein the hSCO has received an injury and comprises glial scar-like tissue and chondroitin sulfate proteoglycans (CSPGs) as a result of the injury.

12. A method of simulating a spinal cord injury, comprising mechanically injuring a human spinal cord organoid (hSCO), wherein:
a) the hSCO comprises neural cells, astroglial cells, and immune cells; or
b) wherein the hSCO comprises neural cells and astroglial cells, and wherein immune cells are added to and infiltrate the hSCO after mechanical injury.

13. The method of claim 12, wherein the immune cells comprise microglia, macrophages, neutrophils, T-cells, or a combination thereof.

14. The method of claim 13, wherein the immune cells comprise microglia.

15. The method of claim 14, wherein the neural cells express class III beta-tubulin (TUJ-1), the microglia express IBA-1, and the astroglial cells express GFAP.

16. The method of claim 12, wherein the hSCO further comprises oligodendrocytes and/or ependymal cells.

17. The method of claim 12, wherein mechanically injuring the hSCO comprises cutting the hSCO or applying a compressive impact force to the hSCO.

18. The method of claim 12, wherein mechanically injuring the hSCO produces a glial scar-like tissue and chondroitin sulfate proteoglycans (CSPGs) on the hSCO.

19. The method of claim 12, further comprising contacting the injured hSCO with a potential therapeutic agent for the treatment of spinal cord injury, and assessing a response of the injured hSCO to the potential therapeutic agent.

20. A method of generating a human spinal cord organoid (hSCO) comprising neural cells, glial cells, and immune cells, comprising:
a) generating hSCOs comprising neural cells and astroglial cells, and
b) co-culturing the hSCO with precursors of immune cells, and inducing differentiation and maturation of the precursor cells.


SEQUENCE LISTING (8 sequences)

SEQ ID NO: 1 - protein, 5 aa, Synthetic construct
IKVAV

SEQ ID NO: 2 - protein, 14 aa, Synthetic construct
VVAAEEEEGIKVAV

SEQ ID NO: 3 - protein, 14 aa, Synthetic construct
AAGGEEEEGIKVAV

SEQ ID NO: 4 - protein, 6 aa, Synthetic construct
VVAAEE

SEQ ID NO: 5 - DNA, 21 bp, Synthetic construct
  source 1..21: mol_type = other DNA
catgtacgttgctatccaggc

SEQ ID NO: 6 - DNA, 21 bp, Synthetic construct
  source 1..21: mol_type = other DNA
ctccttaatgtcacgcacgat

SEQ ID NO: 7 - DNA, 19 bp, Synthetic construct
  source 1..19: mol_type = other DNA
tctgcactgtacccggcta

SEQ ID NO: 8 - DNA, 22 bp, Synthetic construct
  source 1..22: mol_type = other DNA
ccctggtaaatgtgaccctttt